SEQUENCE LISTING (20 sequences)

SEQ ID NO: 1 - DNA, 1509 bp, Ustilago maydis
  misc_feature (1)..(1509): cts1 from Ustilago maydis
atgtttggacgtcttaagcacaggatgtctcgcgctcgactagacgacgacggcaaaaagtcatcgtcatccgcttcatcgttgccaccttctccaaccaaggcagcaacagcctctgctgctggttcggtccctcagactccgactgctacggcacccgaagcttccacgccctcatcctcgacccaacctgagtctcccgtcgcttcggcccccagcagcacttctcctccgagtacgacacctaccacaccagcttccaacacaacccctgcaagcgagatccaaaacaacatcgactcgcaaggtcacgatttcaccaccaatggtgcggtcgtccctcgtgtcaacctcgcctactttacaaactggggtatctacggtcgcaagtactctcctctggacgttccttactgcaacctgacgcacgttctctacgctttcgccgacgtcaaccccgacaccggcgagtgcttcctcaccgatctctgggccgacgagcaaattcactacactggcgactcctggaacgatacgggtaacaacctctacggcaactttaagcaattccttttgctcaagaagaagaaccgtgcgctcaagctcatgctgtccgttggtggatggacatttggtccgcactttgcacctatggcagccgacgccaagaagcgagccaagtttgtctcgactgccatcacgattttggagaacgacgggttggatggaatcgacatcgactgggagtatccctcggacagcacgcaagctgccaactttgttctgctgctcaaagagcttcgtgccggcttgactgcacaccaggccaagaagaacgagaccaacccgtacctactgtccatcgctgcgccctgtggacctgaccattacaaggtgctccaagtagccaagatggaccagtacctggatttctggaacctcatggcgtacgatttcgccggttcgtggtccgcactcaccggccaccaagccaacctgtggaacatcaagggtgcaccgccttcggccgacgattcgatcaactactatattggccagggcgtcgtttcgcacaagctcgttctcggaatcccgctctacggccgtggcttcgaaaacacggacggtccacaacagccgtacaggggtacagggcagggcacgtgggaagccggcaactgggactacaagtttttgcctgtcaaaggtgcaaaggagatgatcaacaccaagatcgctgctagttggagttacgactcagccaagcgcgagttcattagttacgacacacctcagaacgtcttgttgaagtgtcagtacatcaggaacaagaggttgaggggtgccatgttctgggagctgagtggtgacgcaaccaagagccagggtggtgctgaaaggagtctcatcgccctcactgccaagaacatgggcacactcgatgccactttgaaccacatcagttaccctttcagcaagtgggacaatgtcaagaacggcctcaagtaa

SEQ ID NO: 2 - protein, 502 aa, Ustilago maydis
  misc_feature (1)..(502): Cts1 from Ustilago maydis
MFGRLKHRMSRARLDDDGKKSSSSASSLPPSPTKAATASAAGSVPQTPTATAPEASTPSSSTQPESPVASAPSSTSPPSTTPTTPASNTTPASEIQNNIDSQGHDFTTNGAVVPRVNLAYFTNWGIYGRKYSPLDVPYCNLTHVLYAFADVNPDTGECFLTDLWADEQIHYTGDSWNDTGNNLYGNFKQFLLLKKKNRALKLMLSVGGWTFGPHFAPMAADAKKRAKFVSTAITILENDGLDGIDIDWEYPSDSTQAANFVLLLKELRAGLTAHQAKKNETNPYLLSIAAPCGPDHYKVLQVAKMDQYLDFWNLMAYDFAGSWSALTGHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 3 - DNA, 305 bp, Ustilago maydis
  misc_feature (1)..(305): ubi1 3′-UTR
caagaagaagttgaagtaagctgtttcgcttttgctcgattgcgattcggatcttttggctcttggtttcttctcaacacacacacacacacacacacacacacacacacacacacacacacacacacacacgcacatctacatatatgcaacacatcgcacaccacacatggcacagtacaagcattgcgcctgcgtgctggagtgcactggcctcgcgcctacacccactggctctgacagcgctcgtttgtctttgtcagttgtttcaaaaccacatgttattcttggttgtgccgtctaga

SEQ ID NO: 4 - protein, 792 aa, Ustilago maydis
  misc_feature (1)..(792): Rrm4 from Ustilago maydis
MSDSIYAPHNKHKLEAARAADAAADDAATVSALVEPTDSTAQASHAAEQTIDAHQQAGDVEPERCHPHLTRPLLYLSGVDATMTDKELAGLVFDQVLPVRLKIDRTVGEGQTASGTVEFQTLDKAEKAYATVRPPIQLRINQDASIREPHPSAKPRLVKQLPPTSDDAFVYDLFRPFGPLRRAQCLLTNPAGIHTGFKGMAVLEFYSEQDAQRAESEMHCSEVGGKSISVAIDTATRKVSAAAAEFRPSAAAFVPAGSMSPSAPSFDPYPAGSRSVSTGSAASIYATSGAAPTHDTRNGAQKGARVPLQYSSQASTYVDPCNLFIKNLDPNMESNDLFDTFKRFGHIVSARVMRDDNGKSREFGFVSFTTPDEAQQALQAMDNAKLGTKKIIVRLHEPKTMRQEKLAARYNAANADNSDMSSNSPPTEARKADKRQSRSYFKAGVPSDASGLVDEEQLRSLSTVVRNELLSGEFTRRIPKVSSVTEAQLDDVVGELLSLKLADAVEALNNPISLIQRISDAREQLAQKSASTLTAPSPAPLSAEHPAMLGIQAQRSVSSASSTGEGGASVKERERLLKAVISVTESGAPVEDITDMIASLPKKDRALALFNPEFLKQKVDEAKDILDITDESGEDLSPPRASSGSAPVPLSVQTPASAIFKDASNGQSSISPGAAEAYTLSTLAALPAAEIVRLANSQSSSGLPLPKADPATVKATDDFIDSLQGKAAHDQKQKLGDQLFKKIRTFGVKGAPKLTIHLLDSEDLRALAHLMNSYEDVLKEKVQHKVAAGLNK

SEQ ID NO: 5 - DNA, 2379 bp, Ustilago maydis
  misc_feature (1)..(2379): rrm4 from Ustilago maydis
atgtccgattcgatttacgccccacataataagcacaagctcgaggcagcgcgagccgctgatgctgccgccgatgatgccgccaccgtcagcgccctcgtcgagccgaccgactccactgcgcaggcaagtcacgctgcggagcaaaccattgatgcgcatcagcaagcaggtgacgtcgaacctgaacgatgccatcctcacctcactcgccccttgctctaccttagcggtgtcgatgccaccatgactgacaaggagctcgccggccttgtctttgatcaagtactccccgtccggctcaaaattgaccgcaccgtgggcgagggccagacagccagcggcaccgtcgagttccagacgctggataaggccgagaaggcttatgctaccgttcgtcctccgatccagctgcgcatcaaccaggacgcctccatcagagagccgcacccctcggccaaaccacgtcttgtcaagcagctgccacctacatcggacgacgcgttcgtatacgacctcttccgtcctttcggtccgctccgacgagctcagtgtctgctcaccaatccagccggcattcacaccggcttcaagggtatggccgtgcttgaattctactctgaacaagacgcccagcgtgccgaatccgagatgcactgcagcgaagtcggcggcaagtcgatctctgttgccattgataccgctacgcgcaaggtcagcgctgctgcagccgagttccgtccctcggctgctgcattcgttccagcgggcagcatgagtccgtcagctccgtcctttgacccttaccctgctggctctcgttccgtttcgacaggctcagccgcttccatctacgcaacttctggcgccgctccgactcacgatacccgcaacggtgctcagaagggagcgcgtgttccgctccagtattcgagccaagcatctacgtatgtcgacccttgcaacctcttcatcaagaacctcgaccccaacatggaatccaatgacctcttcgacaccttcaaaaggtttggccacatcgtctctgcacgcgtcatgcgcgacgataacggcaagagcagagaatttgggtttgtcagcttcaccactcctgacgaagcgcaacaagctctacaagcaatggacaatgccaaacttggcaccaaaaagatcattgtccgtctgcacgagcccaagacgatgcgtcaggagaagctcgcagcgcgttacaacgctgccaatgccgataacagtgacatgagcagcaacagtccgcccacagaggctcgcaaagccgacaagcgtcaaagcagaagctacttcaaagcaggtgtgccttcggatgccagcggtcttgtagacgaagagcaattgcgctcgctcagcactgtcgtgagaaacgagctcttgtctggcgaatttacacgaagaatccccaaggtttcctcggtcaccgaggcgcagttggacgacgttgtaggcgagctgctcagtctcaagcttgctgatgccgtcgaggcgctcaataacccaatttcgcttattcaaaggatcagcgatgctcgcgagcagcttgcacagaagtctgcctccactctgacggctccgagtccggcgccactgtctgctgaacacccagctatgctcggtattcaggctcagcgtagcgtatcaagcgcatcttcgaccggcgaaggaggtgcgtcggtcaaggagagggagaggctactcaaggctgtcatctccgtcaccgaaagtggtgcccccgttgaggacatcaccgacatgatcgcaagcctgccaaagaaggatcgtgcccttgcgctcttcaatccagagtttctcaagcaaaaagtggacgaagccaaggacattctcgacatcaccgacgaaagtggcgaagatctttcgcctcctcgtgcttccagtgggagcgctccggtgccattgtcagtgcagacgcctgcttctgctattttcaaagatgccagcaacggccagagctccatctcgcctggcgctgctgaggcttacacattatccacgcttgctgcgcttcctgcagccgagattgtgcgcttggccaactcccaatccagctccggtctgcctctccctaaagcggatccagcgacggtcaaggcaaccgacgatttcatcgactcgcttcagggcaaagcagcgcacgatcagaagcagaagctgggtgatcagctcttcaaaaagatccgtacgttcggcgtcaaaggcgcacccaagctcaccatccacttgttggattccgaagacttgcgggcgctggcgcatctcatgaactcgtacgaggacgtgctcaaggaaaaagttcagcacaaggttgctgcaggtctgaacaagtga

SEQ ID NO: 6 - protein, 460 aa, Ustilago maydis
  misc_feature (1)..(470): Cts1 from Ustilago maydis lacking amino acids 1-42 of SEQ ID NO2
SVPQTPTATAPEASTPSSSTQPESPVASAPSSTSPPSTTPTTPASNTTPASEIQNNIDSQGHDFTTNGAVVPRVNLAYFTNWGIYGRKYSPLDVPYCNLTHVLYAFADVNPDTGECFLTDLWADEQIHYTGDSWNDTGNNLYGNFKQFLLLKKKNRALKLMLSVGGWTFGPHFAPMAADAKKRAKFVSTAITILENDGLDGIDIDWEYPSDSTQAANFVLLLKELRAGLTAHQAKKNETNPYLLSIAAPCGPDHYKVLQVAKMDQYLDFWNLMAYDFAGSWSALTGHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 7 - protein, 400 aa, Ustilago maydis
  misc_feature (1)..(400): Cts1 from Ustilago maydis lacking amino acids 1-102 of SEQ ID NO2
GHDFTTNGAVVPRVNLAYFTNWGIYGRKYSPLDVPYCNLTHVLYAFADVNPDTGECFLTDLWADEQIHYTGDSWNDTGNNLYGNFKQFLLLKKKNRALKLMLSVGGWTFGPHFAPMAADAKKRAKFVSTAITILENDGLDGIDIDWEYPSDSTQAANFVLLLKELRAGLTAHQAKKNETNPYLLSIAAPCGPDHYKVLQVAKMDQYLDFWNLMAYDFAGSWSALTGHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 8 - protein, 268 aa, Ustilago maydis
  misc_feature (1)..(268): Cts1 from Ustilago maydis lacking amino acids 1-234 of SEQ ID NO2
ILENDGLDGIDIDWEYPSDSTQAANFVLLLKELRAGLTAHQAKKNETNPYLLSIAAPCGPDHYKVLQVAKMDQYLDFWNLMAYDFAGSWSALTGHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 9 - protein, 184 aa, Ustilago maydis
  misc_feature (1)..(184): Cts1 from Ustilago maydis lacking amino acids 1-318 of SEQ ID NO2
FAGSWSALTGHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 10 - protein, 42 aa, Ustilago maydis
  misc_feature (1)..(42): Cts1 from Ustilago maydis lacking amino acids 1-460 of SEQ ID NO2
SQGGAERSLIALTAKNMGTLDATLNHISYPFSKWDNVKNGLK

SEQ ID NO: 11 - protein, 145 aa, Ustilago maydis
  misc_feature (1)..(145): Cts1 from Ustilago maydis lacking amino acids 104-460 of SEQ ID NO2
MFGRLKHRMSRARLDDDGKKSSSSASSLPPSPTKAATASAAGSVPQTPTATAPEASTPSSSTQPESPVASAPSSTSPPSTTPTTPASNTTPASEIQNNIDSQGSQGGAERSLIALTAKNMGTLDATLNHISYPFSKWDNVKNGLK

SEQ ID NO: 12 - protein, 469 aa, Ustilago maydis
  misc_feature (1)..(469): Cts1 from Ustilago maydis lacking amino acids 200-232 of SEQ ID NO2
MFGRLKHRMSRARLDDDGKKSSSSASSLPPSPTKAATASAAGSVPQTPTATAPEASTPSSSTQPESPVASAPSSTSPPSTTPTTPASNTTPASEIQNNIDSQGHDFTTNGAVVPRVNLAYFTNWGIYGRKYSPLDVPYCNLTHVLYAFADVNPDTGECFLTDLWADEQIHYTGDSWNDTGNNLYGNFKQFLLLKKKNRAITILENDGLDGIDIDWEYPSDSTQAANFVLLLKELRAGLTAHQAKKNETNPYLLSIAAPCGPDHYKVLQVAKMDQYLDFWNLMAYDFAGSWSALTGHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 13 - protein, 491 aa, Ustilago maydis
  misc_feature (1)..(491): Cts1 from Ustilago maydis lacking amino acids 237-247 of SEQ ID NO2
MFGRLKHRMSRARLDDDGKKSSSSASSLPPSPTKAATASAAGSVPQTPTATAPEASTPSSSTQPESPVASAPSSTSPPSTTPTTPASNTTPASEIQNNIDSQGHDFTTNGAVVPRVNLAYFTNWGIYGRKYSPLDVPYCNLTHVLYAFADVNPDTGECFLTDLWADEQIHYTGDSWNDTGNNLYGNFKQFLLLKKKNRALKLMLSVGGWTFGPHFAPMAADAKKRAKFVSTAITILWEYPSDSTQAANFVLLLKELRAGLTAHQAKKNETNPYLLSIAAPCGPDHYKVLQVAKMDQYLDFWNLMAYDFAGSWSALTGHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 14 - protein, 492 aa, Ustilago maydis
  misc_feature (1)..(492): Cts1 from Ustilago maydis lacking amino acids 319-328 of SEQ ID NO2
MFGRLKHRMSRARLDDDGKKSSSSASSLPPSPTKAATASAAGSVPQTPTATAPEASTPSSSTQPESPVASAPSSTSPPSTTPTTPASNTTPASEIQNNIDSQGHDFTTNGAVVPRVNLAYFTNWGIYGRKYSPLDVPYCNLTHVLYAFADVNPDTGECFLTDLWADEQIHYTGDSWNDTGNNLYGNFKQFLLLKKKNRALKLMLSVGGWTFGPHFAPMAADAKKRAKFVSTAITILENDGLDGIDIDWEYPSDSTQAANFVLLLKELRAGLTAHQAKKNETNPYLLSIAAPCGPDHYKVLQVAKMDQYLDFWNLMAYDHQANLWNIKGAPPSADDSINYYIGQGVVSHKLVLGIPLYGRGFENTDGPQQPYRGTGQGTWEAGNWDYKFLPVKGAKEMINTKIAASWSYDSAKREFISYDTPQNVLLKCQYIRNKRLRGAMFWELSGDATKSQGGAERSLIALTAKNMGTLDATLNHISYPFSKWDNVKNGLK

SEQ ID NO: 15 - protein, 79 aa, Ustilago maydis
  misc_feature (1)..(79): amino acids 237-315 of SEQ ID NO2 (Cts1 from Ustilago maydis)
ENDGLDGIDIDWEYPSDSTQAANFVLLLKELRAGLTAHQAKKNETNPYLLSIAAPCGPDHYKVLQVAKMDQYLDFWNLM

SEQ ID NO: 16 - protein, 31 aa, Ustilago maydis
  misc_feature (1)..(31): amino acids 286-316 of SEQ ID NO2 (Cts1 from Ustilago maydis)
LSIAAPCGPDHYKVLQVAKMDQYLDFWNLMA

SEQ ID NO: 17 - protein, 503 aa, Sporisorium reilianum
  misc_feature (1)..(503): endochitinase
MFGRLKHKLSRRFDEDKKSSSPASSLPPSPTKPAAFSAAATTSGSNTAATTPAAPVINTPEATKPSSSTGGATTPVATTPSTAPTTPPATSVDHNTDSQTTDADGHDFTTNGAVVPRVNLGYFTNWGIYGRKYSPLDVPICNLTHILYAFADVNPDTGECILTDLWADEQLHYTGDSWNDAGNNLYGNFKQFLLLKKKNRALKLMLSVGGWTFGPHFAPMAADAKKRAKFVSSAITILENDGLDGIDIDWEYPANDAQAANFVLLLKELRAGLTAHQKKKNDMVPYLLSIAAPCGPDHYKVLQVAKMDPYLDFWNLMAYDFAGSWSTVTGHQANLWNIKGAPPSADDAVNYYIGNGVVSHKLVLGIPLYGRGFENTDGPQQPYKGTGQGTWEAGNWDYKFLPVKGAKEMINTKIAASWSYDSSKREFISYDTPQNVLLKCAYIKQKRLRGAMFWELSGDATKAQGGADRSLVALTAKNMGTLDTTLNHISYPYSKWDNVRAFK

SEQ ID NO: 18 - DNA, 773 bp, Artificial
  codon-optimized anti-myc scFv antibody
ccatggccgaggtcgacctcgtcgagtcgggcggtgacctcgtcaagcccggtggctcgctcaagctctcgtgcgccgcctcgggcttcaccttctcgcactacggcatgtcgtgggtgcgtcagacgcccgacaagcgtctcgagtgggtcgccaccatcggctcgcgtggtacctacacccactaccccgactcggtcaagggtcgcttcaccatctcgcgcgacaacgacaagaacgctctctacctccagatgaactcgctcaagtcggaggacaccgccatgtactactgcgctcgtcgttccgagttctactactacggcaacacgtactactactcggcgatggactactggggtcagggcaccaccgtcaccgtctcgtcgggcggtggtggctcgggcggtggtggctcgggcggtggtggctcggacatcgtgctcacccagtcgcccgcctcgctcgccgtctcgctcggccagcgtgccaccatctcgtgccgtgcctcggaatcggtcgacaactacggcttctcgttcatgaactggttccagcagaagcccggtcagccgcccaagctgctcatctacgccatcagcaaccgcggctcgggcgtccccgcccgtttctcgggctcgggctcgggcaccgacttttcgctcaacatccaccccgtcgaggaggacgaccccgccatgtacttctgccagcagaccaaggaggtgccctggacctttggcgccggtaccaagctcgagatcaagactagt

SEQ ID NO: 19 - protein, 257 aa, Artificial
  Synthetic Construct
MAEVDLVESGGDLVKPGGSLKLSCAASGFTFSHYGMSWVRQTPDKRLEWVATIGSRGTYTHYPDSVKGRFTISRDNDKNALYLQMNSLKSEDTAMYYCARRSEFYYYGNTYYYSAMDYWGQGTTVTVSSGGGGSGGGGSGGGGSDIVLTQSPASLAVSLGQRATISCRASESVDNYGFSFMNWFQQKPGQPPKLLIYAISNRGSGVPARFSGSGSGTDFSLNIHPVEEDDPAMYFCQQTKEVPWTFGAGTKLEIKTS

SEQ ID NO: 20 - protein, 509 aa, Ustilago hordei
  misc_feature (1)..(509): endochitinase
MIFAGLKHKLSRRFDEDKKSSSLASSLPPSPTKPSAYSTAAATDGTAAGSAPAAVAPSSSSNAATPVVTPGTEASNPTAPSTAPTTPPATAAPATDVNQDPENYVADSEGHDFTTNGAVVPRVNLAYFTNWGIYGRKYGPNDVPHCSLTHILYAFADVNPETGDCFLTDLWADEQIHYAGDSWNDRGNNLYGNFKQFLLMKKKNRALKLMLSIGGWTFGPHFAPMAADPKKRARFVTTAIAILENDGLDGLDIDWEYPANAAQASNFTTLLKELRAGLTAHAAKKRDMVPYLLSIAAPCGEQMKTLEVAKMDPYLDFWNLMAYDFAGSWSAVTGHQANLWNIKGKVPSADNAVNFYISNGVVSHKIVLGIPLYGRGFENTNGPQQPYNGTGQGTWEAGNWDYKFLPVKGAKEMINTKIGASWSYDSAKREFISYDTPENVLIKCNYIKQKRLRGAMFWEISGDATKSQGGAERSLVALTAKNMGTLEATLNHISYPFSKWDNVKAGMHK

(19) United States
(12) Patent Application Publication
(10) Pub. No.: US 2014/0227727 A1
(43) Pub. Date: Aug. 14, 2014

(54) UNCONVENTIONAL PROTEIN SECRETION

(71) Applicant: HEINRICH-HEINE-UNIVERSITAET DUESSELDORF, Duesseldorf (DE)

(72) Inventor: Michael Feldbruegge, Duesseldorf (DE)

(21) Appl. No.: 14/349,999

(22) PCT Filed: Oct. 4, 2012
(86) PCT No.: PCT/EP2012/069603

§ 371 Date: Apr. 4, 2014

(30) Foreign Application Priority Data
Oct. 5, 2011 (EP) 11183957.7

(51) Int. Cl. C12N 15/80; C12Q 1/34

(52) U.S. Cl. CPC C12N 15/80; C12Q 1/34
USPC 435/18; 435/320.1; 435/254.11; 435/471; 435/69.1

(57) ABSTRACT

The present invention relates to an expression cassette comprising a nucleotide sequence encoding an amino acid sequence, a fragment or variant thereof which directs unconventional protein secretion and a nucleotide sequence encoding a protein of interest. Also contemplated is a vector which comprises the expression cassette, host cells comprising the vectors as well as methods and uses for the production of a polypeptide of interest.

The present invention relates to an expression cassette comprising a nucleotide sequence encoding an amino acid sequence, a fragment or variant thereof which directs unconventional protein secretion and a nucleotide sequence encoding a protein of interest. Also contemplated is a vector which comprises the expression cassette, host cells comprising the vectors as well as methods and uses for the production of a polypeptide of interest.

The general field of fundamental and applied biotechnology becomes increasingly important for the production of biologicals for human and veterinary use, by using prokaryotic and eukaryotic microorganisms. There are two main systems available for the expression of recombinant proteins; prokaryotic (bacterial), and eukaryotic (yeast, fungal or mammalian). Prokaryotic expression systems have several advantages including, cost, culture conditions, rapid cell growth, yield and relatively short expression time.

However, if the protein is required for functional or enzymatic studies, prokaryotic systems may not be the most suitable, as many proteins will form insoluble aggregates known as inclusion bodies which after refolding may not retain their biological function. Furthermore, bacterial expression systems do not allow for any post-translational modifications to be made (e.g. phosphorylation) which may be necessary for biological activity. Eukaryotic expression systems such as yeast, fungal, mammalian or baculovirus cells are often selected for eukaryotic genes, even when expressed under the control of prokaryotic vectors. The main reason is that bacterial cells are unlikely to recognise human or eukaryotic promoters and terminators. Furthermore prokaryotic cells frequently recognise the protein products of cloned eukaryotic genes as foreign and remove them. Prokaryotes do not carry out the same kind of post-translational modifications as eukaryotes for example, a protein normally coupled to sugars in a eukaryotic cell will be expressed as a ‘naked’ protein when cloned in a bacterial cell. The stability and/or activity of the protein may be affected as a result of this.

For many applications it is preferred that proteins, especially heterologous proteins, are adequately secreted. For this purpose it is necessary that they can pass the cell plasma membrane in reasonable amounts and without substantial loss of protein activity. Secretion of a protein is usually achieved by the use of signal sequences. Specifically, proteins equipped with a signal sequence are secreted through the conventional endoplasmic reticulum (ER)-Golgi secretory pathway, i.e., the conventional secretion pathway. Specifically, from the ER, proteins are transported to the extracellular space or the plasma membrane through the ER-Golgi secretory pathway.

Although the ER-Golgi system is an extremely efficient and accurate molecular machine of protein export, two types of non-conventional protein transport to the cell surface of eukaryotic cells have been discovered: these processes are known as unconventional protein secretion (Nickel. & Seedorf (1992) Annu. Rev. Cell Dev. Biol. 24:287-308). On the one hand, signal-peptide-containing proteins, such as yeast heat-shock protein 150 (Hsp150), the cystic fibrosis transmembrane conductance regulator (CFTR), CD45, the yeast protein Ist2 and the Drosophila melanogaster α integrin subunit, are inserted into the ER but reach the cell surface in a coat protein complex II (CopII) machinery- and/or Golgi independent manner. On the other hand, cytoplasmic and nuclear proteins that lack an ER-signal peptide have been shown to exit cells through ER- and Golgi independent pathways. Such proteins include fibroblast growth factor 2 (FGF2), β-galactoside-specific lectins, galectin 1, galectin 3, certain members of the interleukin family, the nuclear proteins HMGB1 and engrailed homeoprotein as well as the recently discovered Dictyostelium discoideum acylco enzyme A-binding protein (AcbA).

Unconventional protein secretion may have some advantages vis-à-vis conventional protein secretion, since proteins subject to unconventional secretion are not processed by ER or Golgi-dependent post-translational modifications. Furthermore, unconventional protein secretion may also be of particular interest, since over-expressed proteins have a tendency to form aggregates in the host cell. In particular, the circumvention of the conventional secretion pathway through the ER whose lumen constitutes an oxidizing milieu for proteins may under certain circumstances be advantageous to obtain “native” proteins. However, the mechanisms and molecular components of unconventional protein secretion are beginning to emerge, but are not yet fully understood. In fact, up to the present invention, no signal sequences which would direct unconventional protein secretion and thus no protein expression systems that provide the option of unconventional protein secretion are available.

Hence, a need exists for identifying and developing protein expression systems useful for the secretion of proteins, in particular for unconventional secretion of proteins. The present invention meets such needs, and further provides other related advantages. Accordingly, the present invention thus provides as a solution to the technical problem the embodiments concerning expression cassettes, vectors, host cells, kits and uses for the expression of proteins. These embodiments are characterized and described herein, illustrated in the Examples, and reflected in the claims.

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Recently, a novel molecular connection between post-transcriptional regulation at the level of mRNA transport along microtubules and efficient secretion of the bacterial-type endochitinase Cts1 from Ustilago maydis was unraveled (Koepke et al. (2011), Mol. Cell. Proteom. doi:10.1074/mcp.M111.011213). By in vivo UV cross-linking and immune precipitation (CLIP) and FISH experiments, it could be demonstrated that the RNA binding protein Rrm4 interacts with cts1 mRNA in vivo and that Rrm4-dependent particles contain cts1 mRNA. However, while it was previously thought that “RNA transport” sequences would be required that Cts1 reaches its site of secretion at the hyphal tips, the present inventor has now found that even in the absence of mRNA transport, Cts1 is secreted indicating that mRNA transport is not essential for localization of the protein in the cell as well as for its secretion.

In detail, the present inventor observed that a fusion protein between the bacterial-type endochitinase Cts1 from Ustilago maydis and glucuronidase (Gus) was, in the absence of RNA transport, secreted probably via the unconventional secretion pathway. In fact, it was shown that when Gus was fused with a conventional signal peptide, though it was secreted, it was inactive. This is so because glycosylated Gus is inactive (Itturiaga et al. (1989), Plant Cell 1(3)81-390). However, a fusion protein between an amino acid sequence derived from Cts 1 and Gus turned out to be active when secreted. This surprising observation can be explained if the fusion protein is secreted via an unconventional protein secretion pathway which, so to say, keeps back the fusion protein from the glycosylation machinery of a host cell.

Thus, the present invention provides an expression system that makes use of unconventional protein secretion by host cells. Specifically, an amino acid sequence derived from the bacterial-type endochintinase Cts1 from Ustilago maydis, a fragment, homolog or variant thereof as described herein is secreted unconventionally. Though the mechanism of unconventional protein secretion is known from mammalian cells, for example, for FGF2, interleukin-1β, galectin 1, or galectin 3 (Nickel and Rabouille (2009), Nat. Rev. Mol. Cell. Biol. 10:148-155), this mechanism is thus far not exploitable. In particular, no common motif or mechanism was thus unraveled for any of these proteins that could then be generally applied for the export of proteins via the unconventional protein secretion pathway. Moreover, it was surprising for the present inventor that the amino acid sequence which directs unconventional protein secretion was not located at the very N-terminal end of the protein from which it is derived, but it is rather located in the direction of the C-terminal end. In fact, amino acid sequences which direct protein secretion are usually located at the very N-terminal end of a protein.

Protein export via the unconventional pathway has several advantages. Indeed, although N-glycosylation is crucial for correct folding and activity of some proteins, many other such as prokaryotic proteins suffer from unwanted glycosylation. Especially in pharmaceutical applications the glycosylation pattern is particularly important as some patterns are highly allergenic for humans like i.e. observed for proteins produced in ascomycetes like P. pastoris and S. cerevisiae (Gerngross (2004), Nat. Biotechnol. 22:1409-1414). Hence, it is often desired to generate aglycosylated proteins. Accordingly, the present invention paves the way for making use of the mechanism of unconventional protein secretion by co-exporting foreign proteins fused to an amino acid sequence that directs unconventional protein secretion, preferably to the culture supernatant.

The mechanism of unconventional protein secretion mediated by the amino acid sequence, fragments or variants thereof as described herein can be exploited to co-export proteins, in particular to the culture supernatant.

Hence, in a first aspect the present invention relates to an expression cassette (also referred to herein sometimes as “expression system” or “system”) comprising
(a) a nucleotide sequence encoding
   (i) an amino acid sequence having amino acids n-502 of the amino acid sequence shown in SEQ ID No:2, wherein n is amino acid position 1 of SEQ ID No:2, or a fragment thereof which directs unconventional protein secretion, or
   (ii) an amino acid sequence which is 60% identical to the amino acid sequence of (i) and which directs unconventional protein secretion; and
(b) a nucleotide sequence encoding a protein of interest,
   wherein nucleotide sequence (a) and (b) are fused in frame.

As an alternative to the amino acid sequence shown in SEQ ID No:2, the amino acid sequence shown in SEQ ID No: 17 or 20 can be used. Thus, all embodiments pertaining to SEQ ID No: 2 as described herein are equally applicable to SEQ ID No: 17 or 20, respectively, mutatis mutandis.

Preferably, nucleotide sequence (b) of the expression cassette of the present invention does not encode green fluorescence protein or β-glucuronidase (Gus).

For avoidance of doubt, the order of nucleotide sequence (i) and (ii) in the expression cassette can be (5′→3′): nucleotide sequence (i) followed by nucleotide sequence (ii) or nucleotide sequence (ii) followed by nucleotide sequence (i). Accordingly, the amino acid sequence which directs unconventional protein secretion is either fused N-terminal or C-terminal to the protein of interest.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001); Ausubel et al., Current Protocols in Molecular Biology, J, Greene Publishing Associates (1992, and Supplements to 2002); Handbook of Biochemistry: Section A Proteins, Vol 11976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

The expression cassettes of the invention that are preferably present in a vector, preferably an expression vector, are designed such that they allow the expression of the incorporated nucleic acid molecule in host cells. For this purpose the expression cassettes usually comprise the necessary regulatory sequences, such as a promoter and/or a transcription termination sequence such as a poly A site. A particularly preferred host cell is a fungal host cell which is preferably capable of filamentous growth in liquid culture.

Any preferred restriction endonuclease site may be incorporated into the expression cassette and/or vector of the invention as described herein below in more detail (see list of commercially available restriction endonucleases in the New England Biolabs catalogue, which is hereby incorporated by reference). Preferably, the expression cassette comprises at least one restriction enzyme recognition site at about the 3′-end and at least one restriction enzyme recognition site at about the 5′-end.

As used herein, an “expression cassette” refers to a contiguous nucleic acid molecule that can preferably be isolated as a single unit and cloned as a single functional expression unit. A functional expression unit, capable of properly driving the expression of an incorporated polynucleotide is thus also referred to as an “expression cassette” herein.

For example, a sequence cassette may be created enzymatically (e.g., by using type I or type II restriction endonucleases, exonucleases, etc.), by mechanical means (e.g., shearing), by chemical synthesis, or by recombinant methods (e.g., PCR). Expression cassettes generally include the following elements (presented in the 5′-3′ direction of transcription): a transcriptional and translational initiation region, a coding sequence for a gene of interest, and a transcriptional and translational termination region functional in the organism where it is desired to express the gene of interest. The expression cassette of the invention comprises preferably at least two elements (a) and (b):
(a) a nucleotide sequence encoding
   (i) an amino acid sequence having amino acids n-502 of the amino acid sequence shown in SEQ ID No:2, wherein n is amino acid position 1 of SEQ ID No:2, or a fragment thereof which directs unconventional protein secretion, or
   (ii) an amino acid sequence which is 60% identical to the amino acid sequence of (i) and which directs unconventional protein secretion; and
(b) a nucleotide sequence encoding a protein of interest,
   wherein nucleotide sequence (a) and (b) are fused in frame.

Preferably, nucleotide sequence (b) does not encode green fluorescence protein or β-glucuronidase (Gus).

In a preferred general embodiment, the two elements (a) (i.e., nucleotide sequence (a)) and (b) (i.e., nucleotide sequence (b)) are in the form of a transcription unit. If so, said transcription unit only comprises elements (a) and (b), i.e., the transcription unit consists of elements (a) and (b). However, said transcription unit is comprised by the expression cassette of the present invention. Accordingly, the present invention preferably relates to an expression cassette comprising a transcription unit only comprising (or consisting of) elements (a) and (b) as described herein. Thus, though less preferred, if the expression cassette of the present invention comprises a transcription unit only comprising (or consisting of) elements (a) and (b), said expression cassette does not comprises nucleotide sequence (c) (or element (c)) as described herein. However, said expression cassette, in addition to comprising a transcription unit as described herein, may comprise nucleotide sequence (d) (element (d)) as described herein.

A “transcription unit” encodes for a protein and does contain not only the sequence such as nucleotide sequence (a) and (b) that will eventually be directly translated into the protein but also regulatory sequences that direct and regulate the synthesis of that protein.

“Nucleotide sequence (a)” or simply “(a)” is also referred to herein as “first nucleotide sequence” or, sometimes it is referred to as “element (a)”. Likewise, “nucleotide sequence (b)” or simply “(b)” and “nucleotide sequence (c)” or simply “(c)” is sometimes also referred to herein as “second nucleotide sequence” or “element (b)” and “third nucleotide sequence” or “element (c)”, respectively. The first and second nucleotide sequence may be from the same organism or source, however, it is preferred that the first nucleotide and the second nucleotide sequence are not from the same organism or source. Put differently, it is preferred that the first nucleotide sequence is from a nucleotide sequence that is different from the second nucleotide sequence. Accordingly, the first and second nucleotide sequences are preferably heterologous to each other.

The terms “5′” and “3′” is a convention used to describe features of a nucleotide sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, nucleotide sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

The term “nucleotide sequence” or “nucleic acid molecule” refers to a polymeric form of nucleotides (i.e. polynucleotide) of at least 10 bases in length which are usually linked from one deoxyribose or ribose to another. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The term “nucleotide sequence” does not comprise any size restrictions and also encompasses nucleotides comprising modifications, in particular modified nucleotides, e.g., as described herein.

In this regard, a nucleic acid being an expression product is preferably a RNA, whereas a nucleic acid to be introduced into a cell is preferably DNA.

The nucleic acid can be in any topological conformation. For instance, the nucleic acid can be single-stranded, double-stranded, triple-stranded, quadruplexed, partially double-stranded, branched, hairpinned, circular, or in a padlocked conformation.

The term “nucleotide sequence” preferably includes single and double stranded forms of DNA or RNA. A nucleic acid molecule of this invention may include both sense and antisense strands of RNA (containing ribonucleotides), cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The nucleotide sequences of the invention are preferably “isolated” or “substantially pure”. An “isolated” or “substantially pure” nucleotide sequence or nucleic acid (e.g., a RNA, DNA or a mixed polymer) is one which is substantially separated from other cellular components that naturally accompany the native polynucleotide in its natural host cell, e.g., ribosomes, polymerases, and genomic sequences with which it is naturally associated. The term embraces a nucleotide sequence or nucleic acid that (1) has been removed from its naturally occurring environment, (2) is not associated with all or a portion of a polynucleotide in which the “isolated nucleotide sequence” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleotide sequence or nucleic acid so described has itself been physically removed from its native environment. For instance, an endogenous nucleotide sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g., by homologous recombination) for the native promoter of a gene in the genome of a human cell, such that this gene has an altered expression pattern. This gene would now become “isolated” because it is separated from at least some of the sequences that naturally flank it.

A nucleotide sequence is also considered “isolated”, if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleotide sequence” includes a nucleic acid integrated into a host cell chromosome at a heterologous site, a nucleic acid construct present as an episome. Moreover, an “isolated nucleotide sequence” can be substantially free of other cellular material, or substantially free of culture medium when produced by recombinant techniques, or substantially free of chemical precursors or other chemicals when chemically synthesized.

When used herein, the phrase “degenerate variant” of a reference nucleotide sequence encompasses nucleotide sequences that can be translated, according to the standard genetic code, to provide an amino acid sequence identical to that translated from the reference nucleotide sequence.

Unless otherwise indicated, a “nucleotide sequence shown in SEQ ID No:X” refers to a nucleotide sequence, at least a portion of which has either (i) the sequence of a portion of which has either (i) the sequence of SEQ ID No:Y, or (ii).

A “polypeptide” refers to a molecule comprising a polymer of amino acids linked together by a peptide bond(s). Said term is herein interchangeably used with the term “protein”. When used herein, the term “polypeptide” or “protein” also includes a “polypeptide of interest” or “protein of interest” which is expressed by the expression cassettes or vectors or can be isolated from the host cells of the invention. Examples of a protein of interest are enzymes more preferably an amylolytic enzyme, a lipolytic enzyme, a proteolytic enzyme, a cellulytic enzyme, an oxidoreductase or a plant cell-wall degrading enzyme; and most preferably an enzyme having an activity selected from the group consisting of aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, and xylanase, growth factors, cytokines, antibodies or functional fragments thereof such as Fab or F(ab)₂ or derivatives of an antibody such as bispecific antibodies (for example, scFvs), chimeric antibodies, humanized antibodies, single domain antibodies such as Nanobodies or domain antibodies (dAbs), or anticalins (lipocalin muteins).

In fact, the present invention demonstrates that unconventional secretion of Cts1 can be applied for biotechnological approaches. Firstly, Cts1 was fused to Gus and it was observed that the active bacterial protein is present in culture supernatants, indicating that Cts1 is able to co-export heterologous proteins. Gus is an excellent example as it is N-glycosylated and thus, inactive when exported by conventional secretion. This indicates that the expression system based on an amino acid sequence derived from Cts1 aid in avoiding N-glycosylation. Although N-glycosylation is crucial for correct folding and activity of some proteins, many other such as prokaryotic proteins suffer from unwanted glycosylation. Especially in pharmaceutical applications the glycosylation pattern is particularly important as some patterns are highly allergenic for humans like i.e. observed for proteins produced in ascomycetes like P. pastoris and S. cerevisiae (Gerngross (2004)), cited herein. Hence, it is often desired to generate aglycosylated proteins.

In other systems, especially in bacteria, huge proteins are often hard to express. In contrast, the expression system of the present invention promotes the secretion of these proteins as Gus activity in supernatants of strains expressing a 173 kDa Gus-Cts1-GTH fusion protein was detected. This indicates that the unconventional secretory mechanism applied by the present invention is able to export huge proteins.

As a second example for Cts1-mediated export of foreign proteins the expression of scFv antibodies was chosen because these antibodies are high valued pharmaceuticals with improved pharmacokinetic properties compared to monoclonal antibodies. Also in this case the presence of the large fusion protein of 93 kDa in the respective host cells was successfully demonstrated.

A “polypeptide” as used herein encompasses both naturally-occurring and non-naturally-occurring proteins, and fragments, mutants, derivatives, variants and analogs thereof. Polypeptides include polypeptides and peptides of any length, including proteins (for example, having more than 50 amino acids) and peptides (for example, having 2-10, 2-20, 2-30, 2-40 or 2-49 amino acids). Polypeptides include proteins and/or peptides of any activity or bioactivity. A “peptide” encompasses analogs and mimetics that mimic structural and thus biological function.

Polypeptides may further form dimers, trimers and higher oligomers, i.e. consisting of more than one polypeptide molecule. Polypeptide molecules forming such dimers, trimers etc. may be identical or non-identical. The corresponding higher order structures are, consequently, termed homo- or heterodimers, homo- or heterotrimers etc. The terms “polypeptide” and “protein” also refer to naturally or non-naturally modified polypeptides/proteins wherein the modification is effected e.g. by glycosylation, acetylation, phosphorylation and the like. Such modifications are well known in the art.

Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) when it exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well-known in the art. As thus defined, “isolated” does not necessarily require that the protein, polypeptide, peptide or oligopeptide so described has been physically removed from its native environment.

The term “polypeptide fragment” or “fragment” of a polypeptide as used herein refers to a polypeptide that has an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long. Fragments have preferably the same biological activity as the full-length polypeptide.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labelling, e.g., with radionuclides, and various enzymatic modifications, as will be readily appreciated by those well skilled in the art. A variety of methods for labelling polypeptides and of substituents or labels useful for such purposes are well-known in the art, and include radioactive isotopes such as ¹²⁵I, ³²P, ³⁵S, and ³H, ligands which bind to labelled antiligands (e.g., antibodies), fluorophores, chemiluminescent agents, enzymes, and antiligands which can serve as specific binding pair members for a labelled ligand. The choice of label depends on the sensitivity required, ease of conjugation with the primer, stability requirements, and available instrumentation. Methods for labelling polypeptides are well-known in the art.

A “polypeptide mutant” or “mutein” refers to a polypeptide whose sequence contains an insertion, duplication, deletion, rearrangement or substitution of one or more amino acids compared to the amino acid sequence of a native or wild type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein. For example, mutein of the polypeptide encoded by nucleotide sequence (a) and/or (b) is envisaged to be comprised by the expression cassette of the invention.

A mutein has at least 70% overall sequence homology to its wild-type counterpart. Even more preferred are muteins having 80%, 85% or 90% overall sequence homology to the wild-type protein. In an even more preferred embodiment, a mutein exhibits 95% sequence identity, even more preferably 97%, even more preferably 98% and even more preferably 99% overall sequence identity. Sequence homology may be measured by any common sequence analysis algorithm, such as Gap or Besffit.

“Percent (%) amino acid sequence identity” with respect amino acid sequences disclosed herein is defined as the percentage of amino acid residues in a candidate sequence that are identical with the amino acid residues in a reference sequence (such as SEQ ID No:2 (Cts1 from U. maydis) or SEQ ID No: 4 (Rrm4 from U. maydis), after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same is true for nucleotide sequences disclosed herein. Specifically, the U. maydis cts1 nucleotide sequence shown in SEQ ID No: 1 or the U. maydis rrm4 nucleotide sequence shown in SEQ ID No: 5 serve as reference sequences in alignments in order to determine the degree of “percent (%) nucleotide sequence identity”.

Preferred amino acid substitutions are those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology-A 4 Synthesis (2nd Edition, E. S. Golub and D. R. Gren, Eds., Sinauer Associates, Sunderland, Mass. (1991)). Stereoisomers (e.g., D-amino acids) of the twenty conventional amino acids, unnatural amino acids such as a, a-disubstituted amino acids, N-alkyl amino acids, and other unconventional amino acids may also be suitable components for polypeptides of the present invention.

Examples of unconventional amino acids include: 4-hydroxyproline, Y-carboxyglutamate, -N,N,N-trimethyllysine, E-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the left-hand direction is the amino terminal direction and the right hand direction is the carboxy-terminal direction, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. Thus, the term “homologous proteins” is defined to mean that the two proteins have similar amino acid sequences). In a preferred embodiment, a homologous protein is one that exhibits at least 60% sequence homology to the wild type protein, more preferred is at least 70% sequence homology. Even more preferred are homologous proteins that exhibit at least 80%, 85% or 90% sequence homology to the wild type protein. In a yet more preferred embodiment, a homologous protein exhibits at least 95%, 97%, 98% or 99% sequence identity. As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity).

In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (1), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A preferred algorithm when comparing a inhibitory molecule sequence to a database containing a large number of sequences from different organisms is the computer program BLAST (Altschul et al. (1990) J Mol. Biol. 215: 403-410; Gish and States (1993) Nature Genet. 3: 266-272; Madden et al. (1996) Meth. Enzymol. 266: 131-141; Altschul et al. (1997) Nucleic Acids Res. 25: 3389-3402; Zhang and Madden (1997) Genome Res. 7: 649-656), especially blastp or tblastn (Altschul et al., 1997). Preferred parameters for Blastp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than Blastp known in the art. For instance, polypeptide sequences can be compared using FASTA, a program in GCG Version 6.1. FASTA provides alignments and percent sequence identity of the regions of the best overlap between the query and search sequences (Pearson, 1990, herein incorporated by reference). For example, percent sequence identity between amino acid sequences can be determined using FASTA with its default parameters (a word size of 2 and the PAM250 scoring matrix), as provided in GCG Version 6.1, herein incorporated by reference).

By a “substantially pure polypeptide” is meant any polypeptide which has been separated from naturally accompanying components. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight. A substantially pure polypeptide may be obtained, for example, by extraction from a natural source (such as a cell); by expression of a recombinant nucleic acid encoding the polypeptide; or by chemically synthesizing the protein. Purity can be measured by any appropriate method such as those described in column chromatography, polyacrylamide gel electrophoresis, or by HPLC analysis. A protein is substantially free of naturally associated components when it is separated from those contaminants which accompany it in its natural state. Thus, a protein which is chemically synthesized or produced in a cellular system different from the cell from which it naturally originates will be substantially free from its naturally associated components.

The embodiments and disclosure provided herein with respect to polypeptides/proteins herein also pertain, mutatis mutandis, to the polypeptide of interest produced in accordance with the invention.

A large number of suitable methods exist in the art to produce polypeptides (or fusion proteins) in the host cells of the invention. Conveniently, the produced protein is harvested from the culture medium, lysates of the cultured host cell or from isolated (biological) membranes by established techniques. For example, the expression cassettes as described herein comprising, inter alia, the nucleotide sequence encoding the protein of interest can be synthesized by PCR and inserted into an expression vector. Subsequently a cell produced with the method of the present invention may be transformed with the expression vector. Thereafter, the cell is cultured to produce/express the desired protein(s), which is/are isolated and purified. For example, the product may be recovered from the host cell and/or culture medium by conventional procedures including, but not limited to, cell lysis, breaking up host cells, centrifugation, filtration, ultra-filtration, extraction or precipitation. Purification may be performed by a variety of procedures known in the art including, but not limited to, chromatography (e.g. ion exchange, affinity, hydrophobic, chromatofocusing, and size exclusion), electrophoretic procedures (e.g., preparative isoelectric focusing), differential solubility (e.g. ammonium sulfate precipitation) or extraction.

“Isolating the compound” refers to the separation of the compound produced during or after expression of the nucleic acid introduced. After disintegrating the cells, various separation methods are known in the art. In the case of proteins or peptides as expression products, said proteins or peptides, apart from the sequence necessary and sufficient for the protein to be functional, may comprise additional N- or C-terminal amino acid sequences. Such proteins are referred to as fusion proteins.

Polypeptides produced according to the method of the present invention depict preferably good stability properties. It is envisaged that the polypeptides are expressed in a functional form and hence in the right conformation. Accordingly, the invention also provides polypeptides obtained by the production method according to the present invention using the expression cassette, vector and/or host cell described herein in detail above.

Preferred examples of a polypeptide of interest are enzymes including biocatalysts, receptors, receptor ligands such as competitors and scavenger receptors, antibodies, therapeutic proteins such as interferons, BMPs, GDF proteins, fibroblast growth factors, peptides such as protein inhibitors, membrane proteins, membrane-associated proteins, peptide/protein hormones, cytokines, peptidic toxins, peptidic antitoxins, and the like. It is envisaged that the polypeptide of interest is processed during and/or after its isolation from the culture medium and/or host cell by enzymatic cleavage which is possible, since the expression cassette may, inter alia, contain a nucleotide sequence which encodes a protease cleavage site. Furthermore, it is envisaged that the polypeptide of interest is processed by post-isolation methods such as pegylation, acetylation, phosphorylation, and the like.

When a polypeptide of interest is expressed in a host cell of the invention, it may be necessary to modify the nucleotide sequence encoding said polypeptide by adapting the codon usage of said nucleotide sequence to meet the frequency of the preferred codon usage of said host cell. As used herein, “frequency of preferred codon usage” refers to the preference exhibited by the host cell of the invention in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a host cell can be calculated by averaging frequency of preferred codon usage in a large number of genes expressed by the host cell. It is preferable that this analysis be limited to genes that are highly expressed by the host cell. The percent deviation of the frequency of preferred codon usage for a synthetic gene from that employed by a host cell is calculated first by determining the percent deviation of the frequency of usage of a single codon from that of the host cell followed by obtaining the average deviation over all codons. As defined herein, this calculation includes unique codons (i.e., ATG and TGG). In general terms, the overall average deviation of the codon usage of an optimized gene from that of a host cell is calculated using the equation 1A=n=1ZXn-YnXn times 100 Z where Xn=frequency of usage for codon n in the host cell; Yn=frequency of usage for codon n in the synthetic gene; n represents an individual codon that specifies an amino acid; and the total number of codons is Z. The overall deviation of the frequency of codon usage, A, for all amino acids should preferably be less than about 25%, and more preferably less than about 10%.

The term “fused in frame” or “in frame” means that two or more nucleotide sequences as described herein such as nucleotide sequence (a) and nucleotide sequence (b) are covalently linked together by 5′-3′ bonds of the sugar backbone of a nucleic acid such that these two or more nucleotide sequences are in the same open reading frame which is transcribed and then translated as one entity. Accordingly, when the mRNA is transcribed from said covalently linked nucleic acid and translated a “fusion protein” is formed, since a ribosome translates the mRNA of these two or more nucleotide sequences as if it were one entity, i.e., the mRNA encodes, so to say, one protein, i.e., a fusion protein. Said term, however, does not exclude that additional nucleotide sequences such as nucleotide sequence (c) or (d) are contained between two nucleotide sequences such as nucleotide sequence (a) and nucleotide sequence (b).

A “fusion protein” thus refers to a polypeptide comprising a first polypeptide or fragment coupled to a second polypeptide or fragment such as a fusion protein having the amino acid sequence encoded by nucleotide sequence (a) and the amino acid sequence encoded by nucleotide sequence (b). Fusion proteins are useful because they can be constructed to contain two or more desired functional elements from two or more different proteins. Preferably, fusion proteins can be produced recombinantly in accordance with the invention by constructing a first nucleic acid sequence which encodes the first polypeptide or a fragment thereof (encoded by nucleotide sequence (a)) in-frame with a second (encoded by nucleotide sequence (s)), third, fourth, fifth, etc. nucleic acid sequence encoding a further protein or peptide and then expressing the fusion protein. Alternatively, but less preferred a fusion protein can be produced chemically by crosslinking the polypeptide or a fragment thereof to another protein.

Preferably, in the expression cassette of the invention nucleotide sequence (a) is fused in frame with the nucleotide sequence (b) or vice versa, i.e. the nucleotide sequence (b) is fused in frame with nucleotide sequence (a). Accordingly, a fusion protein is formed during translation that comprises (N-terminal) a polypeptide which directs unconventional protein secretion and (C-terminal) a polypeptide of interest; or vice versa, i.e. a fusion protein comprising (N-terminal) a polypeptide of interest and (C-terminal) a polypeptide which directs unconventional protein secretion.

However, while it is envisaged that nucleotide sequence (a) and (b) or (b) and (a) can be directly fused, i.e., no additional nucleotides are between these nucleotide sequence, nucleotide sequence (a) and (b) or (b) and (a) do not have to be directly fused with each other, i.e., without additional nucleotides. Thus, the nucleotide sequence (c) can be in between the nucleotide sequence (a) and (b) or (b) and (a). If so, the nucleotide sequence does not necessarily need to be in frame with the nucleotide sequence (a) and (b) or (b) and (a). Accordingly, nucleotide sequence (c) can be located 5′ and/or 3′ of nucleotide sequence (a) and/or (b).

However, nucleotide sequence (c) can preferably be in frame with nucleotide sequence (a) and (b) or (b) and (a). Thus, it is preferred that the nucleotide sequences (a), (b) and (c) as referred to herein, are fused in frame.

In yet a further preferred embodiment of the invention, nucleotide sequence(s) (c) is/are comprised in the nucleotide sequence (a) and/or (b). Accordingly, one or more nucleotides of the nucleotide sequence (a) and/or (b) may need to be changed so as to conform with nucleotide sequence (c).

More specifically, either the nature of the nucleotide sequence (a) and/or (b) is such that it comprises per se, i.e., due to its nucleotide composition one or more nucleotide sequences (c) or the nucleotide sequence (a) and/or (b) is modified such that it then comprises one or more nucleotide sequence(s) (c). For example, the codon usage can be modified by means and methods known in the art or as is described herein elsewhere. Namely, it is known that some of the naturally-occurring amino acids are encoded by one or more nucleotide triplets and this fact can be exploited when modifying nucleotide sequence (a) and/or (b) so as to then comprise per se one or more nucleotide sequence(s) (c).

In a further preferred aspect of the invention, the expression cassette further comprises one or more (i.e., two, three, four, five, six and more) further nucleotide sequence(s) (c) fused to the 5′- and/or 3′-end of the nucleotide sequence (a) and/or (b). This preferred embodiment, without being bound by theory, may enhance the binding and/or the transport of the resulting transcript (mRNA).

Nucleotide sequence (c) is characterized in that it is bound by a polypeptide comprising at least one sequence specific RNA binding domain. More preferably, the polypeptide which binds nucleotide sequence (c) comprises two, more preferably three, even more preferably four, five, six or more sequence specific RNA binding domains.

A “sequence specific RNA binding domain”, when used herein, is a domain of a protein that binds mRNA, in particular a specific sequence of an mRNA. More preferably, a sequence specific RNA binding domain applied in the invention is of the RNA recognition motif (RRM) type. Preferably, an RRM type comprises two tandem RRM and optionally a further RRM separated from the tandem RRM by a hinge region. More preferably, a sequence specific RNA binding domain comprises the following consensus sequence (L/I)(Y/F/I)(L/V/I)XX(V/L)—32-46—(T/K)GX(G/A)FVXF.

Particularly preferred is a sequence specific RNA binding domain comprising the sequence from amino acids 74-368 of SEQ ID No.4:

(SEQ ID NO: 4)
Met Ser Asp Ser Ile Tyr Ala Pro His Asn Lys His Lys Leu Glu Ala
Ala Arg Ala Ala Asp Ala Ala Ala Asp Asp Ala Ala Thr Val Ser Ala
Leu Val Glu Pro Thr Asp Ser Thr Ala Gln Ala Ser His Ala Ala Glu
Gln Thr Ile Asp Ala His Gln Gln Ala Gly Asp Val Glu Pro Glu Arg
Cys His Pro His Leu Thr Arg Pro Leu Leu Tyr Leu Ser Gly Val Asp
Ala Thr Met Thr Asp Lys Glu Leu Ala Gly Leu Val Phe Asp Gln Val
Leu Pro Val Arg Leu Lys Ile Asp Arg Thr Val Gly Glu Gly Gln Thr
Ala Ser Gly Thr Val Glu Phe Gln Thr Leu Asp Lys Ala Glu Lys Ala
Tyr Ala Thr Val Arg Pro Pro Ile Gln Leu Arg Ile Asn Gln Asp Ala
Ser Ile Arg Glu Pro His Pro Ser Ala Lys Pro Arg Leu Val Lys Gln
Leu Pro Pro Thr Ser Asp Asp Ala Phe Val Tyr Asp Leu Phe Arg Pro
Phe Gly Pro Leu Arg Arg Ala Gln Cys Leu Leu Thr Asn Pro Ala Gly
Ile His Thr Gly Phe Lys Gly Met Ala Val Leu Glu Phe Tyr Ser Glu
Gln Asp Ala Gln Arg Ala Glu Ser Glu Met His Cys Ser Glu Val Gly
Gly Lys Ser Ile Ser Val Ala Ile Asp Thr Ala Thr Arg Lys Val Ser
Ala Ala Ala Ala Glu Phe Arg Pro Ser Ala Ala Ala Phe Val Pro Ala
Gly Ser Met Ser Pro Ser Ala Pro Ser Phe Asp Pro Tyr Pro Ala Gly
Ser Arg Ser Val Ser Thr Gly Ser Ala Ala Ser Ile Tyr Ala Thr Ser
Gly Ala Ala Pro Thr His Asp Thr Arg Asn Gly Ala Gln Lys Gly Ala
Arg Val Pro Leu Gln Tyr Ser Ser Gln Ala Ser Thr Tyr Val Asp Pro
Cys Asn Leu Phe Ile Lys Asn Leu Asp Pro Asn Met Glu Ser Asn Asp
Leu Phe Asp Thr Phe Lys Arg Phe Gly His Ile Val Ser Ala Arg Val
Met Arg Asp Asp Asn Gly Lys Ser Arg Glu Phe Gly Phe Val Ser Phe
Thr Thr Pro Asp Glu Ala Gln Gln Ala Leu Gln Ala Met Asp Asn Ala
Lys Leu Gly Thr Lys Lys Ile Ile Val Arg Leu His Glu Pro Lys Thr
Met Arg Gln Glu Lys Leu Ala Ala Arg Tyr Asn Ala Ala Asn Ala Asp
Asn Ser Asp Met Ser Ser Asn Ser Pro Pro Thr Glu Ala Arg Lys Ala
Asp Lys Arg Gln Ser Arg Ser Tyr Phe Lys Ala Gly Val Pro Ser Asp
Ala Ser Gly Leu Val Asp Glu Glu Gln Leu Arg Ser Leu Ser Thr Val
Val Arg Asn Glu Leu Leu Ser Gly Glu Phe Thr Arg Arg Ile Pro Lys
Val Ser Ser Val Thr Glu Ala Gln Leu Asp Asp Val Val Gly Glu Leu
Leu Ser Leu Lys Leu Ala Asp Ala Val Glu Ala Leu Asn Asn Pro Ile
Ser Leu Ile Gln Arg Ile Ser Asp Ala Arg Glu Gln Leu Ala Gln Lys
Ser Ala Ser Thr Leu Thr Ala Pro Ser Pro Ala Pro Leu Ser Ala Glu
His Pro Ala Met Leu Gly Ile Gln Ala Gln Arg Ser Val Ser Ser Ala
Ser Ser Thr Gly Glu Gly Gly Ala Ser Val Lys Glu Arg Glu Arg Leu
Leu Lys Ala Val Ile Ser Val Thr Glu Ser Gly Ala Pro Val Glu Asp
Ile Thr Asp Met Ile Ala Ser Leu Pro Lys Lys Asp Arg Ala Leu Ala
Leu Phe Asn Pro Glu Phe Leu Lys Gln Lys Val Asp Glu Ala Lys Asp
Ile Leu Asp Ile Thr Asp Glu Ser Gly Glu Asp Leu Ser Pro Pro Arg
Ala Ser Ser Gly Ser Ala Pro Val Pro Leu Ser Val Gln Thr Pro Ala
Ser Ala Ile Phe Lys Asp Ala Ser Asn Gly Gln Ser Ser Ile Ser Pro
Gly Ala Ala Glu Ala Tyr Thr Leu Ser Thr Leu Ala Ala Leu Pro Ala
Ala Glu Ile Val Arg Leu Ala Asn Ser Gln Ser Ser Ser Gly Leu Pro
Leu Pro Lys Ala Asp Pro Ala Thr Val Lys Ala Thr Asp Asp Phe Ile
Asp Ser Leu Gln Gly Lys Ala Ala His Asp Gln Lys Gln Lys Leu Gly
Asp Gln Leu Phe Lys Lys Ile Arg Thr Phe Gly Val Lys Gly Ala Pro
Lys Leu Thr Ile His Leu Leu Asp Ser Glu Asp Leu Arg Ala Leu Ala
His Leu Met Asn Ser Tyr Glu Asp Val Leu Lys Glu Lys Val Gln His
Lys Val Ala Ala Gly Leu Asn Lys

Further preferred proteins with a sequence specific RNA binding domain are Rrm4 from Sporisorium relianum (CBQ73718.1), Coprinopsis cinerea (XP_{—}001832566.2), Laccaria bicolor (XP_{—}001881076.1) and Schizophyllum commune (XP_{—}003027868.1).

A preferred polypeptide comprising at least one sequence specific RNA binding domain that is applied in the invention is one which has at least 60%, more preferably at least 70%, even more preferred at least 80%, particularly preferred at least 90% and even more particularly preferred at least 95% identity to the amino acid sequence shown in SEQ ID No:4. A more preferred polypeptide comprising a sequence specific RNA binding domain that is applied in the invention is shown in SEQ ID No:4.

Likewise, a polypeptide comprising at least one sequence specific RNA binding domain that is applied in the invention, can be encoded by a nucleotide sequence which has at least 60%, 70%, 80%, 90% or 95% identity to the nucleotide sequence shown in SEQ ID No:5 or a fragment thereof and which encodes a protein which is capable of sequence specific RNA binding. Alternatively, a nucleotide sequence can be applied which hybridizes to the nucleotide sequence shown in SEQ ID No:5 or a fragment which encodes a protein which is capable of sequence specific RNA binding.

In a preferred aspect, nucleotide sequence (c) bound by a polypeptide comprising at least one sequence specific RNA binding domain comprises one or more (C/A)(C/A)(C/A) repeats, preferably CAA and/or CA, more preferably CA repeats.

In a more preferred embodiment nucleotide sequence (c) comprises the nucleotide sequence shown in SEQ ID No:3 (3′ UTR of the ubi1 gene of Ustilago maydis:

(SEQ ID NO: 3)
caagaagaag ttgaagtaag ctgtttcgct tttgctcgat tgcgattcgg atcttttggc
tcttggtttc ttctcaacac acacacacac acacacacac acacacacac acacacacac
acacacacac acgcacatct acatatatgc aacacatcgc acaccacaca tggcacagta
caagcattgc gcctgcgtgc tggagtgcac tggcctcgcg cctacaccca ctggctctga
cagcgctcgt ttgtctttgt cagttgtttc aaaaccacat gttattcttg gttgtgccgt
ctaga

If this sequence is fused in frame with the nucleotide sequence(s) of (a) and/or (b), the skilled person will be aware of the fact that this sequence must not have an “in frame stop codon”.

The nucleotide sequence (a) comprises the coding sequence for an amino acid sequence which directs unconventional protein secretion. Generally, the expression “coding sequence” refers to the region of continuous sequential DNA triplets encoding a protein, polypeptide or peptide sequence.

Proteins which are secreted via the unconventional secretion pathway do not use the classical ER-Golgi pathway. Rather, these proteins are secreted through the unconventional secretion pathway which includes various mechanisms. Following endoplasmic reticulum (ER) translocation, signal-peptide-containing proteins are packaged into coat protein complex II (CopII)-coated vesicles that fuse directly with the plasma membrane (mechanism 1). Alternatively, they can fuse with an endosomal or lysosomal compartment (such as late endosomes) that, in turn, fuses with the plasma membrane (mechanism 2). Proteins can also be packaged into non-CopII-coated vesicles that can fuse directly with the plasma membrane (mechanism 3) or can be targeted to the Golgi apparatus (mechanism 4) before reaching the plasma membrane (see Nickel and Rabouille (2009), Nat Rev Mol Cell Biol. 10:148-155). Without being bound by theory, any one of the aforementioned mechanisms 1-4 or all of them is/are envisaged to be used by the host cell of the invention when secreting a protein via the unconventional secretion pathway.

A fragment of the expression cassette of the present invention encoded by nucleotide sequence (a) having amino acids n-502 of the amino acid sequence shown in SEQ ID No:2, wherein n is amino acid position 1 of SEQ ID No:2 comprises preferably at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or 501 amino acids of the amino acid sequence of SEQ ID No:2. Preferably, these aforementioned at least 10 to 501 amino acids are contiguous amino acids.

In the alternative, n may also be amino acid position 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of SEQ ID No:2.

Preferably, a fragment of the expression cassette of the present invention comprises at least amino acids 1-462, 1-463, 1-464, 1-465, 1-466, 1-467, 1-468, 1-469, 1-470, 1-471, 1-472, 1-473, 1-474, 1-475, 1-476, 1-477, 1-478, 1-479, 1-480, 1-481, 1-482, 1-483, 1-484, 1-485, 1-486, 1-487, 1-488, 1-489, 1-490, 1-491, 1-492, 1-493, 1-494, 1-495, 1-496, 1-497, 1-498, 1-499, 1-500 or 1-501 of SEQ ID No:2.

In a preferred embodiment of the expression cassette of the present invention, n is an integer in the range of amino acid position 43 to amino acid position 461 of SEQ ID No:2. Accordingly, for example, nucleotide sequence (a) encodes amino acid positions 43-502, 44-502, 45-502 . . . 461-502 of SEQ ID No:2.

In another preferred embodiment of the expression cassette of the present invention, n is an integer in the range of amino acid position 103 to amino acid position 461 of SEQ ID No:2. Accordingly, the amino acid sequence which directs unconventional protein secretion comprises amino acids n-502 of the amino acid sequence shown in SEQ ID No:2, wherein n is an integer in the range of amino acid position 103 to amino acid position 461 of SEQ ID No:2. By way of example, n can be amino acid position 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, or 461. Accordingly, for example, nucleotide sequence (a) encodes amino acid positions 103-502, 104-502, 105-502 . . . 461-502 of SEQ ID No:2.

Indeed, the present inventor investigated Cts1 secretion using bacterial Gus as a reporter enzyme. In tobacco cells it has been observed, that conventionally secreted Gus is N-glycosylated and thus inactive (Iturriaga et al. (1989), cited herein. In the present invention these observations were confirmed, however in a totally different context, by using Gus fused to the signal peptide of a secreted invertase. By contrast, active Gus was observed in culture supernatants when fusing it to the amino terminus of Cts1. This result indicates that Cts1 is exported by an unconventional mechanism. Secreted proteins usually carry discrete topogenic sequences, the secretion signals, at their N-terminal end which target the proteins to the ER. Furthermore, many other targeting signals e.g., for import to mitochondria or peroxisomes are located at the N-terminus of the respective proteins (Stroud and Walter (2000), cited herein). The present invention demonstrates that the N-terminus of Cts1 is dispensable for secretion in that an N-terminally truncated Cts1₁₀₃₋₅₀₂ variant is still secreted, suggesting that Cts1 does not carry a conventional secretion signal. This is a new finding as other chitinases were shown to harbor N-terminal secretion signals (Adams (2004), Microbiology. 150(Pt 7):2029-35) and the observation is consistent with the results gained with the Gus reporter system. Hence, it is all the more surprising that an amino acid sequence which directs unconventional protein secretion is not located at the very N-terminal end of an endochitinase. Thus, the skilled person would not have had expected the “unusual” localization of such an amino acid sequence in Cts1 from Ustilago maydis.

In another preferred embodiment of the expression cassette of the present invention, n is an integer in the range of amino acid position 235 to amino acid position 461 of SEQ ID No:2. Accordingly, for example, nucleotide sequence (a) encodes amino acid positions 235-502, 236-502, 237-502 . . . 461-502 of SEQ ID No:2.

In another preferred embodiment of the expression cassette of the present invention, n is an integer in the range of amino acid position 319 to amino acid position 461 of SEQ ID No:2. Accordingly, for example, nucleotide sequence (a) encodes amino acid positions 319-502, 320-502, 321-502 . . . 461-502 of SEQ ID No:2.

It is thus a preferred embodiment that the expression cassette of the present invention comprises nucleotide sequence (a) which encodes
(i) amino acids 43-502 of the amino acid sequence shown in SEQ ID No:2 (see SEQ ID No: 6),
(ii) amino acids 103-502 of the amino acid sequence shown in SEQ ID No:2 (see SEQ ID No: 7),
(iii) amino acids 235-502 of the amino acid sequence shown in SEQ ID No:2 (see SEQ ID No: 8),
(iv) amino acids 319-502 of the amino acid sequence shown in SEQ ID No:2 (see SEQ ID No: 9), or
(v) amino acids 461-502 of the amino acid sequence shown in SEQ ID No:2 (see SEQ ID No: 10).

In a preferred alternative embodiment, n is an integer in the range of amino acid position 43, 103, 235, or 319, respectively, to amino acid position 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, or 493. Accordingly, for example, a fragment of the expression cassette of the present invention comprises at least amino acids 462-502, 463-502, 464-502, 465-502, 466-502, 467-502, 468-502, 469-502, 470-502, 471-502, 472-502, 473-502, 474-502, 475-502, 476-502, 477-502, 478-502, 479-502, 480-502, 481-502, 482-502, 483-502, 484-502, 485-502, 486-502, 487-502, 488-502, 489-502, 490-502, 491-502, 492-502, 493-502 of SEQ ID No:2.

Preferably, a fragment (in general) or a fragment defined by amino acid positions (with respect to SEQ ID No:2) of the expression cassette of the present invention directs unconventional protein secretion.

Preferably, the amino acid sequence (encoded by nucleotide sequence (a)) which directs unconventional protein secretion comprises amino acids n-502 of the amino acid sequence shown in SEQ ID No:2, wherein n is amino acid position 103, 235, 319 or 461 of SEQ ID No:2. Yet, n may also be amino acid position 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, or 493 of SEQ ID No:2.

In a preferred embodiment, the nucleotide sequence (a) encoding the amino acid sequence which directs unconventional protein secretion as described herein lacks the nucleotide sequence encoding amino acids 104-460 (see SEQ ID No:11), 200-232 (see SEQ ID No:12), 237-247 (see SEQ ID No:13) and/or 319-328 (see SEQ ID No:14) of the amino acid sequence shown in SEQ ID No:2. This means that, though the afore-mentioned amino acid stretches are lacking, the remaining amino acids are in the form of a fusion protein, i.e., the indicated amino acid stretch is deleted “in frame”

In other preferred embodiments, the nucleotide sequence (a) encoding the amino acid sequence which directs unconventional protein secretion as described herein such as the amino acid sequence that comprises amino acids n-502 of the amino acid sequence shown in SEQ ID No:2, further comprises at its 5′ end a nucleotide sequence encoding amino acids 43-102 of the amino acid sequence shown in SEQ ID No:2. This means that said amino acid sequence additionally comprises at its N-terminus amino acids 43-102 fused to the amino acid sequence that comprises amino acids n-502 of the amino acid sequence shown in SEQ ID No:2, wherein n is an integer in the range of amino acid position 103 to amino acid position 461 of SEQ ID No:2, but which lacks amino acids 319-328 of the amino acid sequence shown in SEQ ID No:2.

In other preferred embodiments, the nucleotide sequence (a) encoding the amino acid sequence which directs unconventional protein secretion as described herein such as the amino acid sequence that comprises amino acids n-502 of the amino acid sequence shown in SEQ ID No:2, wherein n is an integer in the range of amino acid position 103 to amino acid position 461 of SEQ ID No:2, further comprises at its 5′ end a nucleotide sequence encoding amino acids 1-102 of the amino acid sequence shown in SEQ ID No:2. This means that said amino acid sequence additionally comprises at its N-terminus amino acids 1-102 fused to the amino acid sequence that comprises amino acids n-502 of the amino acid sequence shown in SEQ ID No:2, wherein n is an integer in the range of amino acid position 103 to amino acid position 461 of SEQ ID No:2, but which lacks amino acids 319-328 of the amino acid sequence shown in SEQ ID No:2.

In other preferred embodiments, the nucleotide sequence (a) encoding the amino acid sequence which directs unconventional protein secretion as described herein such as the amino acid sequence that comprises amino acids n-502 of the amino acid sequence shown in SEQ ID No:2, wherein n is an integer in the range of amino acid position 103 to amino acid position 461 of SEQ ID No:2, lacks the nucleotide sequence encoding amino acids 104-460, amino acids 200-232 and/or amino acids 237-247 of the amino acid sequence shown in SEQ ID No:2. This means that, though the afore-mentioned amino acid stretches are lacking, the remaining amino acids are in the form of a fusion protein, i.e., the amino acid stretch is deleted “in frame.

In an alternative preferred embodiment, the nucleotide sequence (a) encodes an amino acid sequence having amino acids 1-502 of the amino acid sequence shown in SEQ ID No:2 which directs unconventional protein secretion as described herein lacks the nucleotide sequence encoding amino acids 104-460, amino acids 200-232 and/or amino acids 237-247 of the amino acid sequence shown in SEQ ID No:2. This means that, though the afore-mentioned amino acid stretches are lacking, the remaining amino acids are in the form of a fusion protein, i.e., the amino acid stretch is deleted “in frame”.

In a preferred general embodiment, the nucleotide sequence (a) comprises (in any event) the nucleotide sequence which encodes amino acids 237-315 (see SEQ ID No:15), more preferably amino acids 286-316 (see SEQ ID No:16) of the amino acid sequence shown in SEQ ID No:2 which directs unconventional protein secretion as described herein. Likewise, in another preferred general embodiment, the nucleotide sequence (a) comprises (in any event) the nucleotide sequence which encodes amino acids 104-460, 200-232 and/or 237-247 of the amino acid sequence shown in SEQ ID No:2 which directs unconventional protein secretion as described herein.

In another preferred embodiment, the nucleotide sequence (a) comprises the nucleotide sequence which encodes amino acids 43-502 (see SEQ ID No:6), 103-502 (see SEQ ID No:7), 235-502 (see SEQ ID No:8), 319-502 (see SEQ ID No:9) or 461-502 (see SEQ ID No:10) of the amino acid sequence shown in SEQ ID No:2 which directs unconventional protein secretion as described herein.

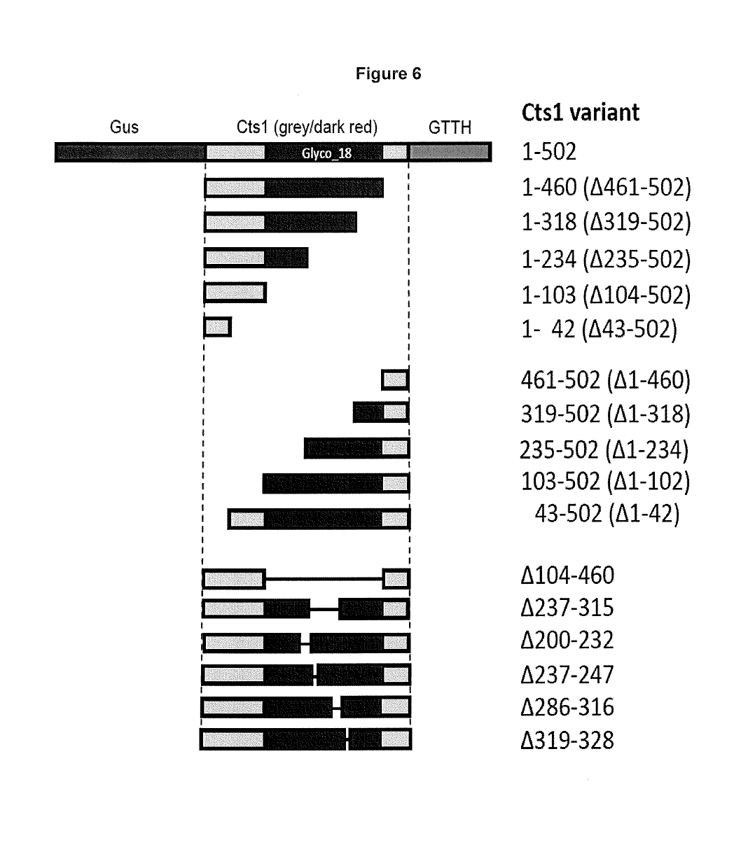

Other preferred fragments which can be applied in the present invention are shown in FIG. 6. FIG. 6 shows the amino acid sequence of Cts1 shown in SEQ ID No:2.

Nucleotide sequence (a) encodes, apart from encoding an amino acid sequence which directs unconventional protein secretion as described herein or a fragment of the amino acid sequence of SEQ ID No:2 which directs unconventional protein secretion, can also encode an amino acid sequence which is at least 60%, preferably at least 70%, more preferably at least 80%, particularly preferably at least 90 or 95% identical to an amino acid sequence derived from SEQ ID No:2 which directs unconventional protein secretion or a fragment of the amino acid sequence of SEQ ID No:2 which directs unconventional protein secretion as described herein. The degree of identity between two amino acid sequences is preferably determined as described herein. Alternatively, a nucleotide sequence (a) can be applied which hybridizes to the nucleotide sequence shown in SEQ ID No:1 or a fragment thereof and which encodes a protein which is secreted via the unconventional secretion pathway.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences (Pearson, 1990, herein incorporated by reference). For instance, percent sequence identity between nucleic acid sequences can be determined using FASTA with its default parameters (a word size of 6 and the NOPAM factor for the scoring matrix) or using Gap with its default parameters as provided in GCG Version 6.1.

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 50%, more preferably 60% of the nucleotide bases, usually at least about 70%, more usually at least about 80%, preferably at least about 90%, and more preferably at least about 95%, 96%, 97%, 98% or 99% of the nucleotide bases, as measured by any well-known algorithm of sequence identity, such as FASTA, BLAST or Gap, as discussed above. Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions”

In the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tm) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., supra, page 9.51, hereby incorporated by reference. For purposes herein, “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65 C for 8-12 hours, followed by two washes in 0.2×SSC, 0.1% SDS at 65° C. for 20 minutes. It will be appreciated by the skilled artisan that hybridization at 65° C. will occur at different rates depending on a number of factors including the length and percent identity of the sequences which are hybridizing.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product. See, e.g., Leung, D. W., et al., Technique, 1, pp. 11-15 (1989) and Caldwell, R. C. & Joyce G. F., PCR Methods Applic., 2, pp. 28-33 (1992)); and “oligonucleotide-directed mutagenesis” (a process which enables the generation of site-specific mutations in any cloned DNA segment of interest. See, e.g., Reidhaar-Olson, J. F. & Sauer, R. T., et al., Science, 241, pp. 53-57 (1988)).

As nucleotide sequence (a) cts1 from Ustilago maydis shown in SEQ ID No.1 or a fragment thereof which encodes a protein secreted via the unconventional pathway is particularly preferred. The nucleotide sequence shown in SEQ ID No.1 encodes the amino acid sequence shown in SEQ ID No.2:

| (SEQ ID NO: 2) | |
|---|---|
| Met Phe Gly Arg Leu Lys His Arg Met Ser Arg Ala Arg Leu Asp Asp | 16 |
| Asp Gly Lys Lys Ser Ser Ser Ser Ala Ser Ser Leu Pro Pro Ser Pro | 32 |
| Thr Lys Ala Ala Thr Ala Ser Ala Ala Gly Ser Val Pro Gln Thr Pro | 48 |
| Thr Ala Thr Ala Pro Glu Ala Ser Thr Pro Ser Ser Ser Thr Gln Pro | 64 |
| Glu Ser Pro Val Ala Ser Ala Pro Ser Ser Thr Ser Pro Pro Ser Thr | 80 |
| Thr Pro Thr Thr Pro Ala Ser Asn Thr Thr Pro Ala Ser Glu Ile Gln | 96 |
| Asn Asn Ile Asp Ser Gln Gly His Asp Phe Thr Thr Asn Gly Ala Val | 112 |
| Val Pro Arg Val Asn Leu Ala Tyr Phe Thr Asn Trp Gly Ile Tyr Gly | 128 |
| Arg Lys Tyr Ser Pro Leu Asp Val Pro Tyr Cys Asn Leu Thr His Val | 144 |
| Leu Tyr Ala Phe Ala Asp Val Asn Pro Asp Thr Gly Glu Cys Phe Leu | 160 |
| Thr Asp Leu Trp Ala Asp Glu Gln Ile His Tyr Thr Gly Asp Ser Trp | 176 |
| Asn Asp Thr Gly Asn Asn Leu Tyr Gly Asn Phe Lys Gln Phe Leu Leu | 192 |
| Leu Lys Lys Lys Asn Arg Ala Leu Lys Leu Met Leu Ser Val Gly Gly | 208 |
| Trp Thr Phe Gly Pro His Phe Ala Pro Met Ala Ala Asp Ala Lys Lys | 224 |
| Arg Ala Lys Phe Val Ser Thr Ala Ile Thr Ile Leu Glu Asn Asp Gly | 240 |
| Leu Asp Gly Ile Asp Ile Asp Trp Glu Tyr Pro Ser Asp Ser Thr Gln | 256 |
| Ala Ala Asn Phe Val Leu Leu Leu Lys Glu Leu Arg Ala Gly Leu Thr | 272 |
| Ala His Gln Ala Lys Lys Asn Glu Thr Asn Pro Tyr Leu Leu Ser Ile | 288 |
| Ala Ala Pro Cys Gly Pro Asp His Tyr Lys Val Leu Gln Val Ala Lys | 304 |
| Met Asp Gln Tyr Leu Asp Phe Trp Asn Leu Met Ala Tyr Asp Phe Ala | 320 |
| Gly Ser Trp Ser Ala Leu Thr Gly His Gln Ala Asn Leu Trp Asn Ile | 336 |
| Lys Gly Ala Pro Pro Ser Ala Asp Asp Ser Ile Asn Tyr Tyr Ile Gly | 352 |
| Gln Gly Val Val Ser His Lys Leu Val Leu Gly Ile Pro Leu Tyr Gly | 368 |
| Arg Gly Phe Glu Asn Thr Asp Gly Pro Gln Gln Pro Tyr Arg Gly Thr | 384 |
| Gly Gln Gly Thr Trp Glu Ala Gly Asn Trp Asp Tyr Lys Phe Leu Pro | 400 |
| Val Lys Gly Ala Lys Glu Met Ile Asn Thr Lys Ile Ala Ala Ser Trp | 416 |
| Ser Tyr Asp Ser Ala Lys Arg Glu Phe Ile Ser Tyr Asp Thr Pro Gln | 432 |
| Asn Val Leu Leu Lys Cys Gln Tyr Ile Arg Asn Lys Arg Leu Arg Gly | 448 |
| Ala Met Phe Trp Glu Leu Ser Gly Asp Ala Thr Lys Ser Gln Gly Gly | 464 |
| Ala Glu Arg Ser Leu Ile Ala Leu Thr Ala Lys Asn Met Gly Thr Leu | 480 |
| Asp Ala Thr Leu Asn His Ile Ser Tyr Pro Phe Ser Lys Trp Asp Asn | 496 |
| Val Lys Asn Gly Leu Lys | 502 |

Either the full-length protein or a fragment thereof which is secreted via the unconventional secretion pathway can be preferably used in the context of the invention. Similarly, the Cts1 from Sporisorium reilianum shown in SEQ ID No: 17 or a fragment thereof is particularly preferred:

(SEQ ID NO: 17)
Met Phe Gly Arg Leu Lys His Lys Leu Ser Arg Arg Phe Asp Glu Asp
Lys Lys Ser Ser Ser Pro Ala Ser Ser Leu Pro Pro Ser Pro Thr Lys
Pro Ala Ala Phe Ser Ala Ala Ala Thr Thr Ser Gly Ser Asn Thr Ala
Ala Thr Thr Pro Ala Ala Pro Val Ile Asn Thr Pro Glu Ala Thr Lys
Pro Ser Ser Ser Thr Gly Gly Ala Thr Thr Pro Val Ala Thr Thr Pro
Ser Thr Ala Pro Thr Thr Pro Pro Ala Thr Ser Val Asp His Asn Thr
Asp Ser Gln Thr Thr Asp Ala Asp Gly His Asp Phe Thr Thr Asn Gly
Ala Val Val Pro Arg Val Asn Leu Gly Tyr Phe Thr Asn Trp Gly Ile
Tyr Gly Arg Lys Tyr Ser Pro Leu Asp Val Pro Ile Cys Asn Leu Thr
His Ile Leu Tyr Ala Phe Ala Asp Val Asn Pro Asp Thr Gly Glu Cys
Ile Leu Thr Asp Leu Trp Ala Asp Glu Gln Leu His Tyr Thr Gly Asp
Ser Trp Asn Asp Ala Gly Asn Asn Leu Tyr Gly Asn Phe Lys Gln Phe
Leu Leu Leu Lys Lys Lys Asn Arg Ala Leu Lys Leu Met Leu Ser Val
Gly Gly Trp Thr Phe Gly Pro His Phe Ala Pro Met Ala Ala Asp Ala
Lys Lys Arg Ala Lys Phe Val Ser Ser Ala Ile Thr Ile Leu Glu Asn
Asp Gly Leu Asp Gly Ile Asp Ile Asp Trp Glu Tyr Pro Ala Asn Asp
Ala Gln Ala Ala Asn Phe Val Leu Leu Leu Lys Glu Leu Arg Ala Gly
Leu Thr Ala His Gln Lys Lys Lys Asn Asp Met Val Pro Tyr Leu Leu
Ser Ile Ala Ala Pro Cys Gly Pro Asp His Tyr Lys Val Leu Gln Val
Ala Lys Met Asp Pro Tyr Leu Asp Phe Trp Asn Leu Met Ala Tyr Asp
Phe Ala Gly Ser Trp Ser Thr Val Thr Gly His Gln Ala Asn Leu Trp
Asn Ile Lys Gly Ala Pro Pro Ser Ala Asp Asp Ala Val Asn Tyr Tyr
Ile Gly Asn Gly Val Val Ser His Lys Leu Val Leu Gly Ile Pro Leu
Tyr Gly Arg Gly Phe Glu Asn Thr Asp Gly Pro Gln Gln Pro Tyr Lys
Gly Thr Gly Gln Gly Thr Trp Glu Ala Gly Asn Trp Asp Tyr Lys Phe
Leu Pro Val Lys Gly Ala Lys Glu Met Ile Asn Thr Lys Ile Ala Ala
Ser Trp Ser Tyr Asp Ser Ser Lys Arg Glu Phe Ile Ser Tyr Asp Thr
Pro Gln Asn Val Leu Leu Lys Cys Ala Tyr Ile Lys Gln Lys Arg Leu
Arg Gly Ala Met Phe Trp Glu Leu Ser Gly Asp Ala Thr Lys Ala Gln
Gly Gly Ala Asp Arg Ser Leu Val Ala Leu Thr Ala Lys Asn Met Gly
Thr Leu Asp Thr Thr Leu Asn His Ile Ser Tyr Pro Tyr Ser Lys Trp
Asp Asn Val Arg Ala Phe Lys

Similarly, the chitinase UHOR 06394 (http://mips.helmholtz-muenchen.de/genre/proj/MUHDB/) from Ustilago hordei shown in SEQ ID No: 20 or a fragment thereof is particularly preferred:

(SEQ ID NO: 20)
Met Ile Phe Ala Gly Leu Lys His Lys Leu Ser Arg Arg Phe Asp Glu
Asp Lys Lys Ser Ser Ser Leu Ala Ser Ser Leu Pro Pro Ser Pro Thr
Lys Pro Ser Ala Tyr Ser Thr Ala Ala Ala Thr Asp Gly Thr Ala Ala
Gly Ser Ala Pro Ala Ala Val Ala Pro Ser Ser Ser Ser Asn Ala Ala
Thr Pro Val Val Thr Pro Gly Thr Glu Ala Ser Asn Pro Thr Ala Pro
Ser Thr Ala Pro Thr Thr Pro Pro Ala Thr Ala Ala Pro Ala Thr Asp
Val Asn Gln Asp Pro Glu Asn Tyr Val Ala Asp Ser Glu Gly His Asp
Phe Thr Thr Asn Gly Ala Val Val Pro Arg Val Asn Leu Ala Tyr Phe
Thr Asn Trp Gly Ile Tyr Gly Arg Lys Tyr Gly Pro Asn Asp Val Pro
His Cys Ser Leu Thr His Ile Leu Tyr Ala Phe Ala Asp Val Asn Pro
Glu Thr Gly Asp Cys Phe Leu Thr Asp Leu Trp Ala Asp Glu Gln Ile
His Tyr Ala Gly Asp Ser Trp Asn Asp Arg Gly Asn Asn Leu Tyr Gly
Asn Phe Lys Gln Phe Leu Leu Met Lys Lys Lys Asn Arg Ala Leu Lys
Leu Met Leu Ser Ile Gly Gly Trp Thr Phe Gly Pro His Phe Ala Pro
Met Ala Ala Asp Pro Lys Lys Arg Ala Arg Phe Val Thr Thr Ala Ile
Ala Ile Leu Glu Asn Asp Gly Leu Asp Gly Leu Asp Ile Asp Trp Glu
Tyr Pro Ala Asn Ala Ala Gln Ala Ser Asn Phe Thr Thr Leu Leu Lys
Glu Leu Arg Ala Gly Leu Thr Ala His Ala Ala Lys Lys Arg Asp Met
Val Pro Tyr Leu Leu Ser Ile Ala Ala Pro Cys Gly Glu Gln Met Lys
Thr Leu Glu Val Ala Lys Met Asp Pro Tyr Leu Asp Phe Trp Asn Leu
Met Ala Tyr Asp Phe Ala Gly Ser Trp Ser Ala Val Thr Gly His Gln
Ala Asn Leu Trp Asn Ile Lys Gly Lys Val Pro Ser Ala Asp Asn Ala
Val Asn Phe Tyr Ile Ser Asn Gly Val Val Ser His Lys Ile Val Leu
Gly Ile Pro Leu Tyr Gly Arg Gly Phe Glu Asn Thr Asn Gly Pro Gln
Gln Pro Tyr Asn Gly Thr Gly Gln Gly Thr Trp Glu Ala Gly Asn Trp
Asp Tyr Lys Phe Leu Pro Val Lys Gly Ala Lys Glu Met Ile Asn Thr
Lys Ile Gly Ala Ser Trp Ser Tyr Asp Ser Ala Lys Arg Glu Phe Ile
Ser Tyr Asp Thr Pro Glu Asn Val Leu Ile Lys Cys Asn Tyr Ile Lys
Gln Lys Arg Leu Arg Gly Ala Met Phe Trp Glu Ile Ser Gly Asp Ala
Thr Lys Ser Gln Gly Gly Ala Glu Arg Ser Leu Val Ala Leu Thr Ala
Lys Asn Met Gly Thr Leu Glu Ala Thr Leu Asn His Ile Ser Tyr Pro
Phe Ser Lys Trp Asp Asn Val Lys Ala Gly Met His Lys

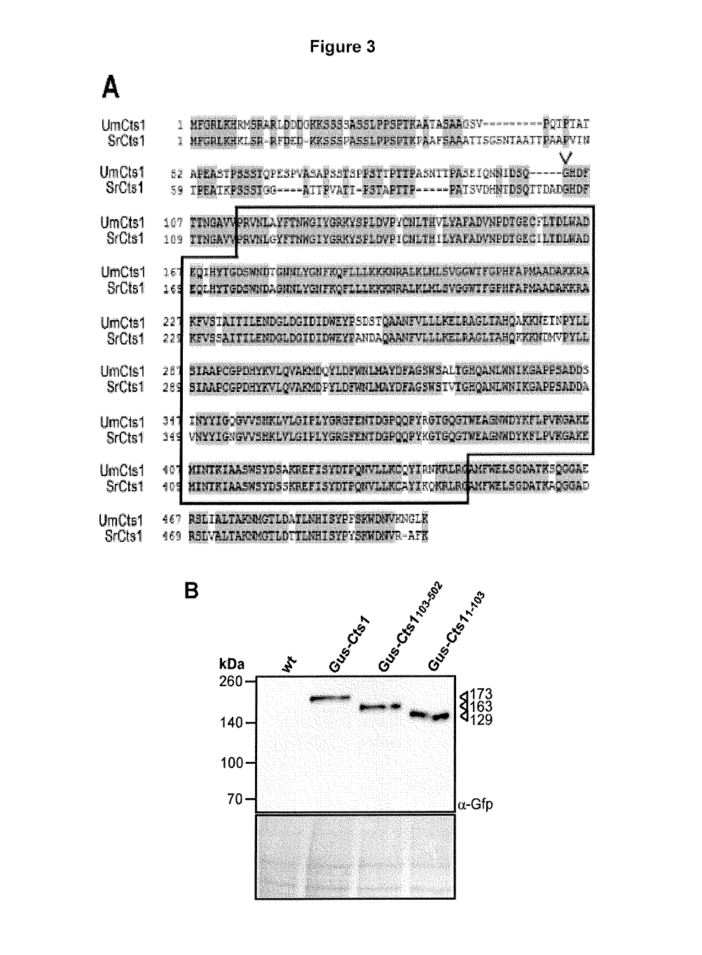

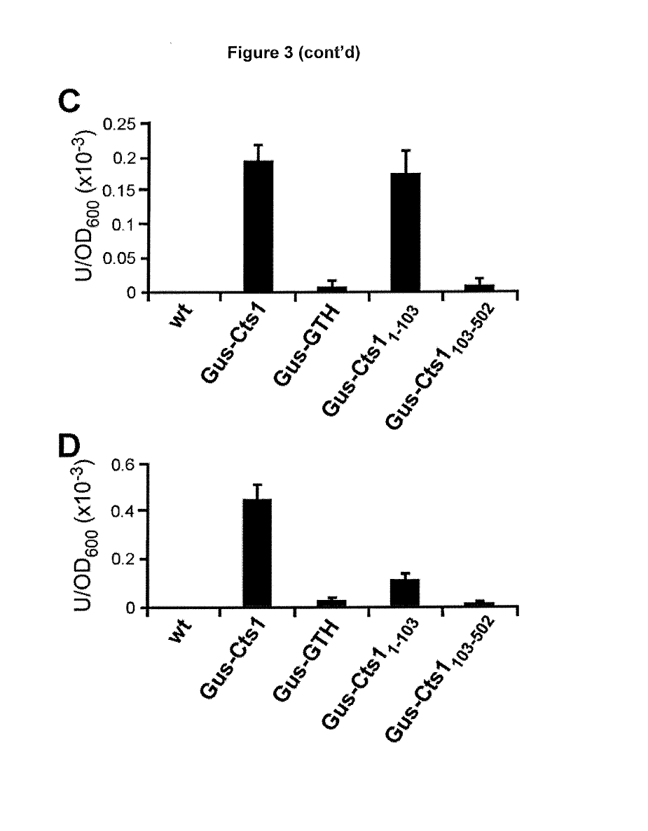

Any fragment of the Cts1 from Sporisorium reilianum shown in SEQ ID No: 17 or Cts1 from Ustilago hordei shown in SEQ ID No: 20 may be applied in the expression cassette of the present invention. Specifically, any fragment as defined herein with reference to SEQ ID No: 2 can be derived from SEQ ID No:17 or 20. In fact, the skilled person is readily in a position to align SEQ ID No:2 and SEQ ID No:17 or 20 and to then find the corresponding fragment by way of corresponding amino acid positions (see FIG. 3A).

Likewise, any other endochitinase that shares identity or homology with Cts1 from Ustilago maydis can be applied in the present invention. The skilled person is readily in a position to identify endochitinases which share identity or homology with Cts1 from Ustilago maydis and can thus readily determine which of the fragments/regions of SEQ ID No:2 as defined herein corresponds to the respective fragment/region of an endochitinase different from Cts1 from Ustilago maydis. Non-limiting examples are sequences from Trametes versicolor or Laccaria bicolor.

The term “position” when used in accordance with the invention means the position of either an amino acid within an amino acid sequence depicted herein or the position of a nucleotide within a nucleic acid sequence depicted herein. The term “corresponding” as used herein also includes that a position is not only determined by the number of the preceding nucleotides/amino acids. Accordingly, the position of a given amino acid in accordance with the invention which may be substituted may very due to deletion or addition of amino acids elsewhere in an endochitinase. Similarly, the position of a given nucleotide in accordance with the present invention which may be substituted may vary due to deletions or additional nucleotides elsewhere in a endochitinase 5′-untranslated region (UTR) including the promoter and/or any other regulatory sequences or gene (including exons and introns).

Thus, under a “corresponding position” in accordance with the invention it is preferably to be understood that nucleotides/amino acids may differ in the indicated number but may still have similar neighbouring nucleotides/amino acids. Said nucleotides/amino acids which may be exchanged, deleted or added are also comprised by the term “corresponding position”.

Specifically, in order to determine whether a nucleotide residue or amino acid residue of the nucleotide or amino acid sequence of an endochitinase different from Cts1 from Ustilago maydis corresponds to a certain position in the nucleotide sequence or the amino acid sequence of another endochitinase, a skilled artisan can use means and methods well-known in the art, e.g., alignments, either manually or by using computer programs such as BLAST2.0, which stands for Basic Local Alignment Search Tool or ClustalW or any other suitable program which is suitable to generate sequence alignments. Accordingly, Cts1 of SEQ ID No:2 can serve as “subject sequence” or “reference sequence”, while the amino acid sequence of another endochitinase different from Cts1 from Ustilago maydis described herein serves as “query sequence”.

Given the above, a skilled artisan is thus readily in a position to determine which amino acid position in Cts1 from Ustilago maydis as described herein corresponds to an amino acid of an endochitinase other than Cts1 from Ustilago maydis. Specifically, a skilled artisan can align the amino acid sequence of Cts1 from Ustilago maydis as described herein, with the amino acid sequence of a different endochitinase to determine which amino acid(s) of said Cts1 from Ustilago maydis correspond(s) to the respective amino acid(s) of the amino acid sequence of said different endochitinase. More specifically, a skilled artisan can thus determine which amino acid position or fragment of the amino acid sequence of said different endochitinase corresponds to the respective amino acid position(s) or fragment of the amino acid sequence of SEQ ID No: 2.

Further preferred proteins or fragments thereof that may effect secretion via the unconventional secretion pathway are Um00501, Um10053, Um02769, Um02175, Um04092, Um01202, Um03294, Um10753, Um12131, whereby Um00501, Um02769, Um02175, Um01202 and Um03294 are preferred. These proteins are available through the Ustilago maydis genome sequencing project at MIPS (http://mips.gsf.de/genre/proj/ustilago). As described herein, further candidate proteins or fragments thereof can be identified via SecretomeP. Preferably, a protein or fragment thereof has a NN-score of more than about 0.6 when said protein or fragment thereof is analysed via the SecretomeP algorithm.

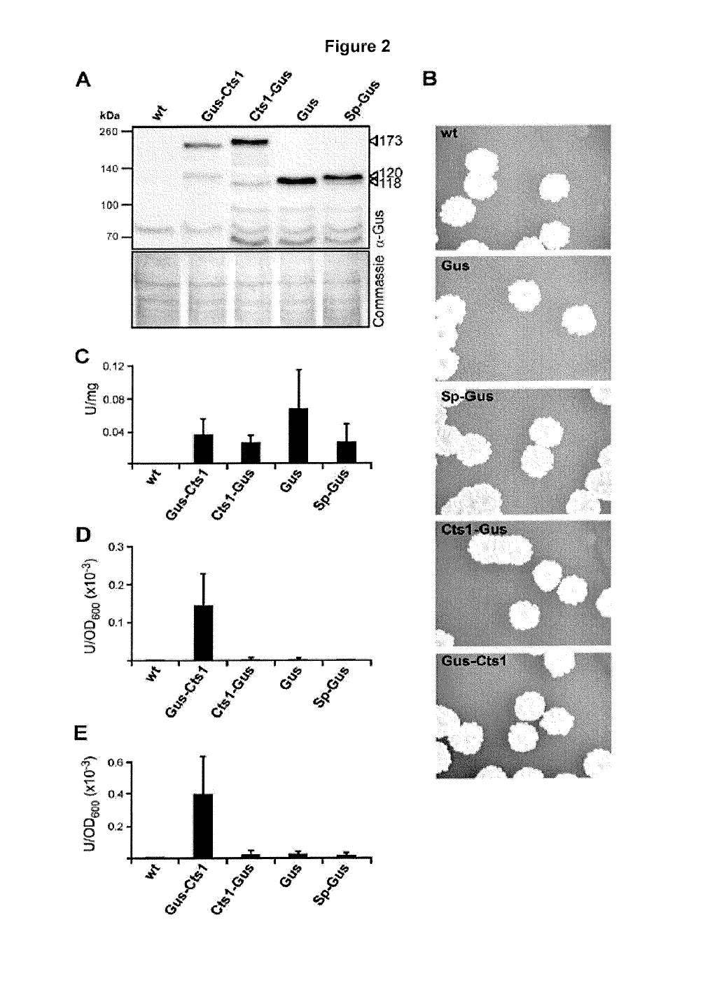

Indeed, as is shown in FIG. 2, C1ts1 effects secretion of a fusion protein via the unconventional secretion pathway. Thus, it is reasonable to assume that proteins or fragments thereof other than Cts1, but which are derived from Cts1 or are identical in a certain degree to Cts1 as described herein will function in the same manner. The present invention provides an easy assay for testing as to whether a protein or fragment thereof can effect secretion of a protein via the unconventional secretion pathway. Namely, a fusion protein between the protein or fragment thereof of interest can be fused with Gus and tested in Ustilago maydis. In fact, if the protein or fragment thereof effects secretion via the unconventional secretion pathway, then Gus should be active in the supernatant. Otherwise, Gus will be glycosylated and will be inactive in the supernatant.

It is preferred that an expression cassette described herein does not comprise a nucleotide sequence (b) which encodes green fluorescence protein (GFP). The term “GFP” also includes enhanced green fluorescence protein (eGFP).

It is preferred that an expression cassette described herein does not comprise a nucleotide sequence (b) which encodes a β-glucuronidase (Gus). Accordingly, for example, a nucleotide sequence encoding a fusion protein between Gus and Cts1 from Ustilago maydis is excluded. Similarly a nucleotide sequence encoding a fusion protein between Gus and Cts1 (lacking intron 1) from Ustilago maydis is excluded.

It is envisaged that a polypeptide which is preferably secreted via the unconventional secretion pathway by a host cell is preferably determined by an in silico analysis, in particular by the absence of a (secretion) signal peptide sequence (SignalP) and/or by the prediction that a protein is extracellularly located (Protcomp) and/or is predicted to be secreted via the unconventional secretion pathway (SecretomeP). Determination of a (secretion) signal sequence is preferably done as described herein above.

Once a protein is identified to be a candidate for secretion via the unconventional secretion pathway, a functional test can be preferably made. Accordingly, for example, Ustilago maydis cells can be used and the candidate protein can be fused in frame with β-glucuronidase (Gus). In case the candidate protein is indeed secreted via the unconventional secretion pathway, Gus is not modified in the ER/Golgi. However, in case the candidate protein is secreted via the conventional pathway, Gus is modified in the ER/Golgi, thereby losing some of its activity (Iturriaga et al. (1989), The Plant Cell 1 (3), 381-390). An assay how to check for secretion via the unconventional secretion pathway is described in the Examples (see “Cts is secreted by an unconventional mechanism”).

In another preferred aspect of the invention, the expression cassette further comprises one or more (i.e., two, three, four, five, six and more) further nucleotide sequence(s) (a) fused in frame to the 5′- and/or 3′-end of the coding region of the nucleotide sequence of (a) and/or (b).

Without being bound by theory, it is assumed that this preferred embodiment may enhance or make secretion more efficiently.

Preferably, the expression cassette of the invention comprising nucleotide sequence (a), (b) and/or (c) further comprise(s) one or more (i.e., two, three, four, five, six and more) further nucleotide sequence(s) (d) fused to the 5′- and/or 3′-end of the nucleotide sequence (a), (b) and/or (c). Accordingly, nucleotide sequence (d) is present “between” nucleotide sequences (a), (b) and/or (c) in the order as referred to herein in (i) to (vi).

In a preferred embodiment, nucleotide sequence (d) is comprised in the nucleotide sequence (a), (b) and/or (c). As described herein in the context of modifying nucleotide sequence (a) and/or (b) such that nucleotide sequence (c) is comprised in these nucleotide sequence(s), the nucleotide sequence (a), (b) and/or (c) can also be modified such that nucleotide sequence (d) is comprised in the nucleotide sequence (a), (b) and/or (c).

Preferably, nucleotide sequence (d) comprises at least 3 nucleotides, e.g., 3, 6, 9, 12, 15, 18, 21, 24, 27, 30 or more nucleotides.

In a preferred embodiment, nucleotide sequence (d) comprises one or more (i.e., two, three, four, five, six and more) restriction enzyme recognition sites. These restriction enzyme recognition sites may be in the form of a “multiple cloning site”, abbreviated as “MCS” and also known as a “polylinker”.

Nucleotide sequence(s) (d) is/are preferably fused in frame with the nucleotide sequence of (a), (b) and/or (c). Accordingly, if nucleotide sequence (d) is fused in frame with the nucleotide sequence of (a), (b) and/or (c), said nucleotide sequence (d) encodes a heterologous polypeptide.

Preferably, said heterologous polypeptide is a linker, tag and/or cleavage site for a protease.

A tag may be used to allow identification and/or purification of the protein of interest Examples of affinity tags that may be used in accordance with the invention include, but are not limited to, HAT, FLAG, c-myc, hemagglutinin antigen, His (e.g., 6×His) tags, flag-tag, strep-tag, strepII-tag, TAP-tag, One-Strep tag, chitin binding domain (CBD), maltose-binding protein, immunoglobulin A (IgA), His-6-tag, glutathione-S-transferase (GST) tag, intein and streptavidie binding protein (SBP) tag. It is also envisaged that said heterologous polypeptide could be a whole immunoglobulin or, preferably any Fc region of an antibody such as FcIgG, FcIgA, FcIgM, FcIgD or FcIgE.

A linker can be a peptide bond or a stretch of amino acids comprising at least one amino acid residue which may be arranged between the components of the fusion proteins in any order. Such a linker may in some cases be useful, for example, to improve separate folding of the individual domains or to modulate the stability of the fusion protein. Moreover, such linker residues may contain signals for transport, protease recognition sequences or signals for secondary modification. The amino acid residues forming the linker may be structured or unstructured. Preferably, the linker may be as short as 1 amino acid residue or up to 2, 3, 4, 5, 10, 20 or 50 residues. In particular cases, the linker may even involve up to 100 or 150 residues.

A cleavage site for a protease may be one for a serine protease, threonine protease, cysteine protease, aspartate protease, metalloprotease and/or glutamic acid protease.

The expression cassette of the invention is preferably driven by an expression control sequence, i.e. its expression is controlled by an expression control sequence which is preferably either a constitutively active or inducible expression control sequence (preferably a promoter) that is operatively linked with the expression cassette.

The expression cassette can be inserted (integrated) into the genome of a host cell or can be propagated in the form of an autonomously replicating element such as a linear DNA or circular plasmid. The plasmid can be a low-copy number plasmid or a high-copy number plasmid. Genomic insertion can be done into a single genomic locus or can be done into one or more genomic loci, i.e., multi-copy insertion. The insertion can be made in the genomic locus of the nucleotide sequence which encodes a protein which directs unconventional protein secretion or it can be made ectopically, i.e., into a genomic locus which is not the genomic locus of the nucleotide sequence which encodes a protein which directs unconventional protein secretion. In case of Ustilago maydis acting as host cell, the insertion is preferably made in the ip-locus commonly known in the art. In the ip-locus a single insertion or multi-copy insertions can be made.

The term “expression” as used herein means the transcription of an expression cassette to produce the corresponding mRNA and translation of this mRNA to produce the corresponding gene product, such as a polypeptide, or protein.

“Operatively linked” expression control sequences refers to a linkage in which the expression control sequence is contiguous with the expression cassette, as well as expression control sequences that act in trans or at a distance to control expression of the expression cassette.

The term “expression control sequence” as used herein refers to polynucleotide sequences which are necessary to affect the expression of the expression cassette o which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion.

The term “control sequences” is intended to include, at a minimum, all components whose presence is essential for expression, and can also include additional components whose presence is advantageous, for example, leader sequences and fusion partner sequences.

A promoter sequence is preferably inserted upstream of the expression cassette and regulates its expression. Promoter sequences are non-coding regulatory sequences for transcription, usually located nearby the start of the coding sequence, which may be referred to as the gene promoter or the regulatory sequence. Put into a simplistic yet basically correct way, it is the interplay of the promoter with various specialized proteins called transcription factors that determine whether or not a given coding sequence may be transcribed and eventually translated into the actual protein encoded by the gene.

It will be recognized by a person skilled in the art that any compatible promoter can be used for recombinant expression in fungal host cells. The promoter itself may be preceded by an upstream activating sequence, an enhancer sequence or combination thereof. These sequences are known in the art as being any DNA sequence exhibiting a strong transcriptional activity in a cell and being derived from a gene encoding an extracellular or intracellular protein. It will also be recognized by a person skilled in the art that termination and polyadenylation sequences may suitably be derived from the same sources as the promoter.

In case of the host cell being Ustilago maydis, a preferred promoter is the constitutive tef, otef promoter (Spellig et al. (1996), Mol Gen Genet 252:503-509), hsp70 promoter (Holden et al., EMBO J. 8:1927-1934. A preferred inducible promoter is the nar1 promoter (Brachmann et al., (2001), Mol Microbiol. 42:1047-63) or the crg1 promoter (Bottin et al. (1996), Mol Gen Genet 253:342-352).

The expression cassette of the invention may further comprise a nucleotide sequence encoding a marker protein. Preferably, said marker protein resistance against an antibiotic or anti-metabolite.

A marker protein, in accordance with the invention, means a protein which provides the transformed cells with a selection advantage (e.g. growth advantage, resistance against an antibiotic) by expressing the corresponding gene product. Marker genes code, for example, for enzymes causing a resistance to particular antibiotics. As used herein, the term “marker gene” refers to a gene whose product confers a characteristic to the cell expressing the marker gene that allows it to be distinguished from cells that do not express the marker gene. In some embodiments, the marker gene allows screening and/or selection of cells. In some such embodiments, the marker gene is a “screenable marker” or a “selectable marker”. Screening and/or selection may be accomplished based on the presence or absence of the marker. In some embodiments, the screenable or selectable marker confers resistance to an agent such as an antibiotic. In some embodiments, the screenable or selectable marker confers an ability that provides an advantage in a particular set of growth conditions over cells that do not express the screenable or selectable marker.

As described above, the selectable marker can be the expression product of a gene encoding a protein restoring prototrophy for an organic compound, also referred to as prototrophy restoring gene. In this case, the selectable marker introduced enables the cell to synthesize said compound by itself so that it is no longer or less dependent on the external supply of said compound with the medium. Accordingly, a prototrophy restoring gene as used in the present invention is a gene encoding an expression product, i.e. the selectable marker, which reduces or preferably abolishes the dependency of the host cell on external supply of an organic compound by facilitating its synthesis in the cell.

Selection for cells expressing said prototrophy restoring gene is carried out by culturing said cells on/in medium not containing said compound. Only cells expressing said prototrophy restoring gene will grow. The expression product of said gene may be a constituent of a synthesis pathway and the product produced by said constituent may have to be further processed in order to obtain the organic compound otherwise externally supplied. Prototrophy restoring genes commonly applied to plant or fungal cells are e.g. those expressing proteins conferring arginine prototrophy, tryptophan prototrophy, uridine prototrophy or genes enabling for nitrate or sulphate utilization. If the selectable marker is the expression product of a prototrophy restoring gene, the selecting agent is the medium in which the cell is cultivated and which does not contain the respective organic compound. Responsiveness in that case is expressed e.g. in growth rates of the cell. Thus, the higher the expression of the selectable marker, the higher the growth rate of the cell in the absence of the respective compound.

For some prototrophy restoring genes, the amount of expression product sufficient to result in prototrophy is very low. Accordingly, it is more laborious to distinguish cells expressing said prototrophy restoring selectable marker at a low level from those that express it at a high level. In order to facilitate said distinction, such a prototrophy restoring gene can be co-introduced together with a nucleic acid encoding a reporter gene the detectability of which is proportional to its expression level. Accordingly, in this embodiment, the selectable marker according to the invention is composed of the auxotrophy gene and the reporter gene.

In case the fungal host cell is an Ustilago maydis cell, preferred marker genes encode a resistance gene against hygromycin, G418, phleomycin, nourseothricin and carboxin.

In a further aspect, the invention relates to a vector comprising the expression cassette described herein.

The term “vector” as used herein refers to a nucleic acid sequence, e.g., DNA derived from a plasmid, cosmid, virus, or synthesized by chemical or enzymatic means, into which the expression cassette may be inserted or cloned, where the nucleic which encode for the nucleotide sequences described herein. Preferably the vector is an expression vector. A typical expression vector contains a promoter element, which mediates the initiation of transcription of mRNA, the protein coding sequence, and signals required for the termination of transcription and polyadenylation of the transcript.

The vector can contain one or more unique restriction sites for this purpose, and may be capable of autonomous replication in a fungal host cell or may be ectopically or homologously integrated. The vector may have a linear, circular, or supercoiled configuration and may be complexed with other vectors or other material for certain purposes. The components of a vector can contain but is not limited to a DNA molecule incorporating DNA; a sequence encoding an excision protein or another desired product; and regulatory elements for transcription, translation, RNA stability, and replication.

The vector may comprise a polylinker (multiple cloning site), i.e. a short segment of DNA that contains many restriction sites, a standard feature on many plasmids used for molecular cloning. Multiple cloning sites typically contain more than 5, 10, 15, 20, 25, or more than 25 restrictions sites. Restriction sites within an MCS are typically unique (i.e., they occur only once within that particular plasmid). MCSs are commonly used during procedures involving molecular cloning or subcloning.

The expression cassette is inserted into the expression vector as a DNA construct. This DNA construct can be recombinantly made from a synthetic DNA molecule, a genomic DNA molecule, a cDNA molecule or a combination thereof. The DNA construct is preferably made by ligating the different fragments to one another according to standard techniques known in the art.

The gene coding for the protein of interest may be part of the expression vector. Preferably, the expression vector is a DNA vector. The vector conveniently comprises sequences that facilitate the proper expression of the expression cassette of the invention. These sequences typically comprise promoter sequences, transcription initiation sites, transcription termination sites, and polyadenylation functions as described herein. Additionally, the vector system may comprise a DNA sequence coding for a selection marker as described herein. Preferably, this selection marker is capable of being incorporated in the genome of the host organism upon transformation, and was not expressed functionally by the host prior to transformation. Transformed host cells can then be selected and isolated from untransformed cells on the basis of the incorporated selection marker.

Hence, according to one embodiment of the present invention the expression vector comprises a predefined restriction site, which can be used for linearization of the vector nucleic acid prior to transfection. Intelligent placement of said linearization restriction site is important, because said restriction site determines where the vector nucleic acid is opened/linearized and thus determines the order/arrangement of the expression cassettes when the construct is integrated into the genome of the fungal host cell.

Vectors used for expressing the expression cassette including the nucleotide sequence coding for the protein of interest usually contain transcriptional control elements suitable to drive transcription such as e.g. promoters, enhancers, polyadenylation signals, transcription pausing or termination signals as elements of an expression cassette. For proper expression of the polypeptides, suitable translational control elements are preferably included in the vector, such as e.g. 5′ untranslated regions leading to 5′ cap structures suitable for recruiting ribosomes and stop codons to terminate the translation process. In particular, the nucleotide sequence serving as the selectable marker genes as well as the nucleotide sequence encoding the protein of interest can be transcribed under the control of transcription elements present in appropriate promoters. The resultant transcripts of the selectable marker genes and that of the protein of interest harbour functional translation elements that facilitate substantial levels of protein expression (i.e. translation) and proper translation termination.

According to one embodiment, the expression cassette(s) for expressing the polypeptide(s) of interest comprise(s) a stronger promoter and/or enhancer than the expression cassettes for expressing the selectable markers. This arrangement has the effect that more transcript for the polypeptide of interest is generated than for the selection markers. It is advantageous that the production of the polypeptide of interest which is secreted is dominant over the production of the selection markers, since the individual cell capacity for producing heterologous proteins is not unlimited and should thus be focused to the polypeptide of interest.

Furthermore, the expression cassettes may comprise an appropriate transcription termination site. This, as continued transcription from an upstream promoter through a second transcription unit may inhibit the function of the downstream promoter, a phenomenon known as promoter occlusion or transcriptional interference. This event has been described in both prokaryotes and eukaryotes. The proper placement of transcriptional termination signals between two transcription units can prevent promoter occlusion. Transcription termination sites are well characterized and their incorporation in expression vectors has been shown to have multiple beneficial effects on gene expression.

Most eukaryotic nascent mRNAs possess a poly A tail at their 3′ end which is added during a complex process that involves cleavage of the primary transcript and a coupled polyadenylation reaction. The polyA tail is advantageous for mRNA stability and transferability. Hence, the expression cassettes of the vector according to the present invention usually comprise a polyadenylation site.

The expression cassettes may comprise an enhancer (see above) and/or an intron. According to one embodiment, the expression cassette(s) for expressing the polypeptide of interest comprise an intron. Usually, introns are placed at the 5′ end of the open reading frame. Accordingly, an intron may be comprised in the expression cassette(s) for expressing the polypeptide(s) of interest in order to increase the expression rate. Said intron may be located between the promoter and or promoter/enhancer element(s) and the 5′ end of the open reading frame of the polypeptide to be expressed. Several suitable introns are known in the state of the art that can be used in conjunction with the present invention

One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a fungal host cell upon introduction into the host cell, and are thereby replicated along with the host genome. Moreover, certain preferred vectors are capable of directing the expression of the expression cassette to which they are operatively linked. Such vectors are referred to herein as “recombinant expression vectors” (or simply, “expression vectors”).

In a further aspect, the invention relates to a (recombinant) fungal host cell (including fungi and yeasts) comprising the expression cassette or the vector described herein. Preferably, the fungal host cell is capable of filamentous growth, preferably in liquid medium. Particularly preferred, the fungal host cell is Ustilago maydis.

The term “recombinant host cell” (or simply “host cell”), as used herein, is intended to refer to a host cell into which a nucleic acid comprising an expression cassette or vector as described herein has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may, for example, be a mammalian cell, an insect cell, a yeast cell, a fungal cell, a bacterial cell. Preferably, said host cell is an isolated hot cell. A particularly preferred host cell is a fungal cell, e.g., an Ustilago maydis cell. Preferably, said host cell can be grown in culture.

In a preferred embodiment, the host cell of the present invention does not secrete proteases which take action on a protein of interest as described herein. Accordingly, a host cell is thus preferably manipulated so that any such proteases are inactivated, e.g., by knock-out or pull-down by, e.g. iRNA, siRNA, etc. In case of a fungal host cell or yeast host cell, the protease that is preferably inactivated is Kex2. Accordingly, Kex2-negative fungal or yeast host cells are preferred. In case of the particularly preferred host cell Ustilago maydis, the protease that is preferably inactivated is Kex 2 encoded by the gene um02843 (http://mips.gsf.de/genre/proj/ustilago). The skilled person is aware of means and methods for inactivating any such protease. In case of Ustilago maydis, Kex2 can, for example, be knocked-out, either fully or partially, e.g., by homologous recombination. Other proteases that are preferably inactivated, either additionally or alternatively to Kex2, in Ustilago maydis are a secreted aspartic protease Um04926, designated Pep4; a lysosomal serine protease Um04400, designated Prb1 and/or a lysosomal tripeptidyl peptidase Um06118, designated TppA (http://mips.gsf.de/genre/proj/ustilago).

It is also a preferred embodiment that in a host cell of the present invention the nucleotide sequence encoding an amino acid sequence having amino acids n-502 of the amino acid sequence shown in SEQ ID No:2 or a homologous sequence thereto such as an ortholog as described herein may be inactivated, e.g., by knock-out (full-length or partially), pull-down or the like. Put differently, it is preferred that the “internal” copy of the nucleotide sequence encoding an amino acid sequence having amino acids n-502 of the amino acid sequence shown in SEQ ID No:2 or a homologous sequence thereto such as an ortholog as described herein may preferably be inactivated.

The term “introducing a nucleic acid” refers to the application of a nucleic acid to fungal cells and its subsequent uptake and incorporation into the genetic information of said cells, in particular in the nucleus.

In general, the genetic alteration of a fungal cell resulting from the introduction/uptake and expression of foreign genetic material is termed “transformation” Yeasts and fungi may be transformed or by commonly known methods. By protoplast transformation, fungal cells can be converted to protoplasts by removing their cell wall, and can then be soaked in a solution containing DNA and transformed to become genetically modified.

The terms “genetically modified” and “transgenic” are used herein interchangeably. A transgenic or genetically modified fungal cell is one that has a genetic background which is at least partially due to manipulation by the hand of man through the use of genetic engineering. For example, the term “transgenic cell”, as used herein, refers to a cell whose DNA contains an exogenous nucleic acid not originally present in the non-transgenic cell. A transgenic cell may be derived or regenerated from a transformed cell or derived from a transgenic cell.

A further aspect of the invention relates to a method for the production of the fungal host cell, said method comprising transforming a fungal cell with the expression cassette or the vector described herein. Likewise, the expression cassette or the vector described herein can be used for the production of a recombinant fungal host cell.

For example, the expression cassette or vector of the invention may either be integrated into the genome (ectopically or homologously) of the fungal cell or it may be maintained in some form extrachromosomally. Autonomously replicating sequences that can be used for the generation of free replicating vectors are, for example, known in Ustilago maydis (Tsukuda et al. (1988), Mol Cell Biol 8: 3703-3709).

In a yet further aspect, the invention concerns a method for the production of a polypeptide of interest comprising
(a) culturing the fungal host cell described herein to allow expression of said polypeptide;
(b) harvesting said polypeptide from the culture medium or fungal host cells such as from the cell wall as described herein.

Likewise, the fungal host cell or vector described herein can be used for the production of a polypeptide of interest.

The invention also features a kit (expression system) comprising the expression cassette, the vector and/or the fungal host cell described herein, and optionally means for transforming a fungal host cell, fungal host cells as such, culture medium and/or an antibiotic or anti-metabolite for selecting and/or growing transformed fungal host cells.

In some embodiments, kits further comprise buffers for carrying out reactions and/or reagents for transforming cells with vectors.

Given the findings of the present inventor that N-glycosylation of a detectable marker protein such as β-glucuronidase (Gus) could be exploited for elucidating as to whether a protein might be secreted via unconventional secretion, it is another aspect of the present invention to provide a method for identifying an amino acid sequence which directs unconventional protein secretion, comprising
(a) providing a host cell expressing a fusion protein comprising (i) an amino acid sequence which is suspected or assumed to direct unconventional protein secretion and (ii) an amino acid sequence encoding a marker protein having a detectable activity which is subject to N-glycosylation via the ER/Golgi-pathway in said host cell, thereby inactivating the detectable activity of said marker protein, and
(b) determining whether said marker protein is secreted by said host cell by detecting its activity,
   wherein said amino acid sequence which is suspected or assumed to direct unconventional protein secretion directs unconventional protein secretion if it is active after secretion by said host cell.

The fusion protein may be designed such that amino acid sequence (i) is N- or C-terminal to amino sequence (ii).

N-glycosylation takes place in the ER and Golgi-apparatus, i.e., in the ER/Golgi-network. Accordingly, if a protein the activity of which would be inactivated by N-glycosylation is subject to N-glycosylation, it will be inactivated. An example for such a protein is β-glucuronidase (Gus). Hence, β-glucuronidase (Gus) is a preferred marker protein for application in the method for identifying an amino acid sequence which directs unconventional protein secretion.

Accordingly, the present invention also relates to the use of β-glucuronidase (Gus) for the identification of an amino acid sequence which directs unconventional protein secretion. When used herein, the gene for Gus is called gusA or uidA.

A “marker protein” may be any protein that has a detectable activity. An “activity” of a marker protein may be enzymatic activity, fluorescence, or bioluminescence. A “detectable” activity is an activity that can be detected, for example, by enzymatic activity, fluorescence activity, or bioluminescence activity. However, an activity of a marker protein may also be detectable by way of binding the protein with an antibody having a detectable label. For example, if a marker protein would be glycosylated at a position which is otherwise accessible for the antibody, the antibody would, after secretion of the then-glycosylated marker protein, not be capable of binding to said glycosylated marker protein. However, if said marker protein would be secreted by unconventional protein secretion directed via an amino acid sequence suspected or assumed to direct unconventional secretion, said antibody could bind said marker protein, thereby detecting the same.

The Figures show:

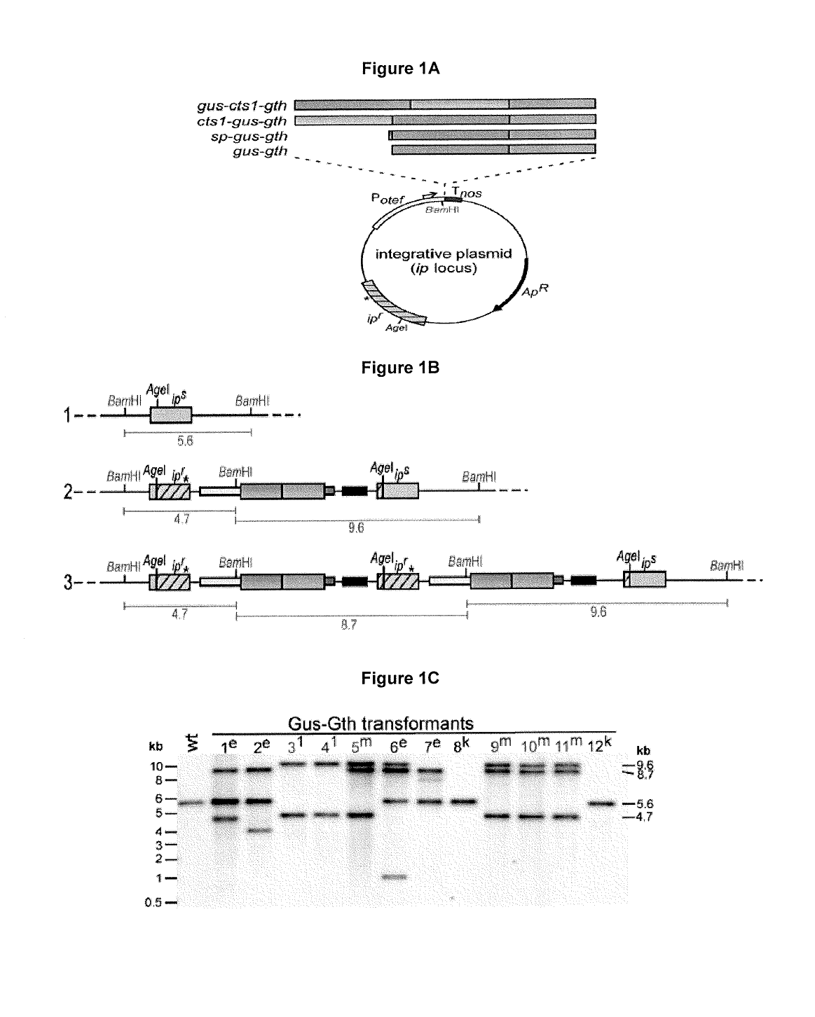

FIG. 1: Generation of reporter strains for unconventional secretion

A, Schematic representation of the reporter constructs generated to confirm unconventional secretion of Cts1-fusion proteins. All four constructs were inserted into a plasmid that contains an ip^{r} allele (red-striped rectangle) for heterologous recombination at the ip locus (see part B). The position of the mutation that leads to the H253L exchange in the Ip^{r} protein is indicated by asterisks (Keon et al. (1991), Curr. Genet. 19(6):475-481; Broomfield and Hargreaves (1992), Cur. Genet. 22(2):117-121). All reporter genes were under control of the constitutive promoter P_{otef} and the transcription termination sequence Tₙₒₛ (Brachmann et al. (2004), Mol. Gen. Genom. 272:216-226). gth, triple tag including sequences that code for the green-fluorescent protein Gfp, a Tap tag and a His tag; sp, sequence encoding the signal peptide of the putative secreted invertase Suc2 (Um01945); ap^{R}, gene mediating ampicillin resistance.

B, Schematic view on the genomic region of the ip locus that can be used for integration of plasmids containing the ip^{r} allele. Organization of the wild type ip locus (ip^{s}) as well as after single or multiple integration of an ip-integrative plasmid containing the fusion gene gus-gth (not to scale). The restriction endonuclease AgeI was used for linearization of the plasmid. 1, wild type; 2, single homolgous recombination; 3, multiple homologous recombination. Grey ip regions are derived from the wild type gene. Red-striped ip regions were located on the integrated plasmid as part of the ip^{r} allele. ip^{s}, wild type gene encoding the iron-sulphur subunit of the succinate dehydrogenase (carboxin sensitive); ip^{r}, mutated ip allele that confers carboxin resistance (Keon et al. (1991); Broomfield and Hargreaves (1992), cited above). For Southern blot analysis BamHI restriction was performed. A 2.07 kb probe covering the complete ip gene was used for detection. Expected fragments are shown in grey below the respective genomic regions.

C, Southern blot of putative Gus-GTH strains that were obtained after transformation of strain AB33 (contains wild type ip allele, wt) with an integrative plasmid (pGus-GTH) containing the fusion gene gusA-gth. gDNA was hydrolized with BamHI and resulting DNA fragments were separated on a 1% TAE agarose gel. Expected fragments were 5.6 kb for the wild type locus, 4.7 and 9.6 kb for single integration of the plasmid and 4.7, 8.7 and 9.6 kb for multiple plasmid integrations. Transformants that integrated the plasmid correctly are numbered in red and labeled with ¹ for single or ^{m} for multiple integrations. Mutants with gene conversions are labeled with ^{k} and ectopic integrations with ^{e}.

FIG. 2: Gus-Cts1 fusion proteins are secreted to the culture supernatant

In all depicted experiments the parental strain AB33 (wt) was used as negative control and all proteins were fused to a GTH tag consisting of Gfp, Tap tag and His tag.

A, Western blot depicting expression of the four Gus-fusion proteins by AB33 (wt) derivatives. 10 μg protein of whole cell extracts was analysed with anti-Gus antibodies. Sp, signal peptide of Suc2 (Um01945). The Commassie Brilliant Blue stained membrane visualizes equal loading. The parental strain AB33 does not express Gus-fusion proteins and was used as a negative control (left lane). Expected band sizes (indicated by asterisks) were 173 kDa for Cts1-Gus-GTH as well as Gus-Cts1-GTH, 118 kDa for Gus-GTH and 120 kDa for Sp-Gus-GTH.

B, Gus activity of the depicted Gus-reporter strains growing in the yeast form was assayed on 5-bromo-4-chloro-3-indolyl-beta-D-glucuronic acid (X-Gluc)-containing plates. All strains are AB33 derivatives. The parental strain is shown on the uppermost picture.

C, Gus activity determined in whole cell extracts of the indicated AB33 derivatives growing in the yeast form. 4-methylumbelliferyl β-D-galactopyranoside (MUG) was used as a substrate. The diagram shows mean values of six biological replicates. Error bars represent standard deviation.

D, Gus activity determined in cell-free culture supernatants of the indicated AB33 derivatives growing in the yeast form. MUG was used as a substrate. The diagram shows mean values of seven biological replicates. Error bars represent standard deviation.

E, Gus activity determined in cell-free culture supernatants of the indicated AB33 derivatives grown in the filamentous form. MUG was used as a substrate. The diagram shows mean values of six biological replicates. Error bars represent standard deviation.

FIG. 3: The N-terminal domain of Cts1 is dispensable for secretion

A, Amino acid alignment of U. maydis Cts1 indicated as UmCts1 (Um10419) (SEQ ID No:2) and the orthologous protein of the close relative Sporisorium reilianum depicted as SrCts1 (Sr15153) (SEQ ID No:17). Identical amino acids are shaded. The predicted Glyco_{—}18 domains of the two proteins (SMART; http://smart.embl-heidelberg.de/) are boxed. The first amino acid of the truncated protein Cts1₁₀₃₋₅₀₂ lacking the N-terminal domain (see B and C) is marked by a red arrowhead.

B, Western blot depicting expression of fusion proteins including the truncated Cts1₁₀₃₋₅₀₂ protein version of about 163 kDa in comparison to the full-length fusion protein of 173 kDa. 10 μg protein of whole cell extracts was analysed with anti-Gfp antibodies. The Commassie Brilliant Blue stained membrane visualizes equal loading. All proteins were fused to a sequence encoding a GTH tag consisting of Gfp, Tap tag and His tag.

C, Gus activity assays of yeast cell culture supernatants comparing the same AB33 derivatives as depicted in B. The diagram shows mean values of three biological replicates. The Error bars represent standard deviation.

D, Gus activity assays of filamentous culture supernatants of the strains described in C. The diagram shows mean values of three biological replicates. The Error bars represent standard deviation.

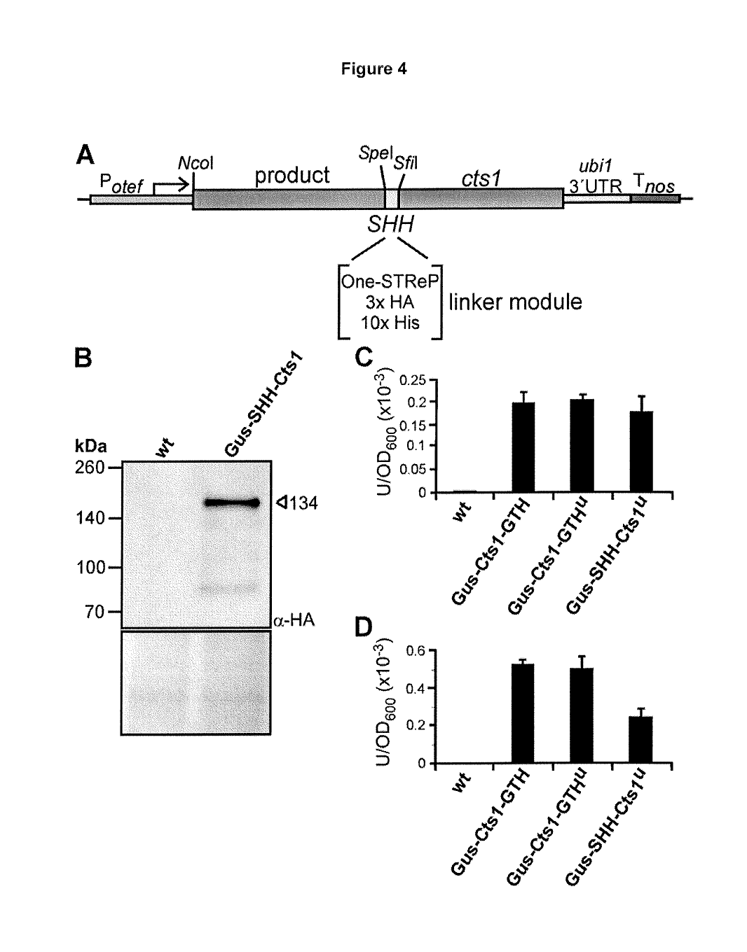

FIG. 4: Rationale for a novel U. maydis expression vector and its application for Cts1-mediated export of foreign proteins

A, View on the schematic architecture of the expression cassette in the integrative vector pRabX1. The cassette allows the expression of N-terminal protein fusions with Cts1. The gene encoding the protein of interest can be inserted in a one-step cloning via NcoI and SpeI. An internal linker encoding different tags for purification and detection of the corresponding fusion protein was inserted. In the depicted version of the expression vector this linker consists of a One-STReP tag (IBA, Göttingen), a triple HA tag and a 10×His-tag (SHH). This linker can easily be exchanged to other cassettes, e.g., comprising protease cleavage sites using SpeI and SfiI restriction. In addition, the cassette harbors a sequence corresponding to the ubi1 3′UTR.

B, Western blot depicting expression of a Gus-SHH-Cts1 fusion protein migrating at the expected size of about 163 kDa in comparison to the progenitor strain AB33 (wt). 10 μg protein of whole cell extracts were analysed with anti-HA antibodies. The Commassie Brilliant Blue stained membrane visualizes equal loading.

C, Gus activity assays of cell-free supernatants of AB33 Gus-SHH-Cts1 yeast cells secreting the Gus-SHH-Cts1fusion protein. As controls, Gus activity of yeast supernatants isolated from progenitor strain AB33 and AB33 Gus-Cts1-GTH were analysed.

D, Gus activity assays of cell-free supernatants of AB33 Gus-SHH-Cts1 filaments secreting the Gus-SHH-Cts1fusion protein. As controls, Gus activity of supernatants isolated from filaments of the progenitor strain AB33 and AB33 Gus-Cts1-GTH were analysed.

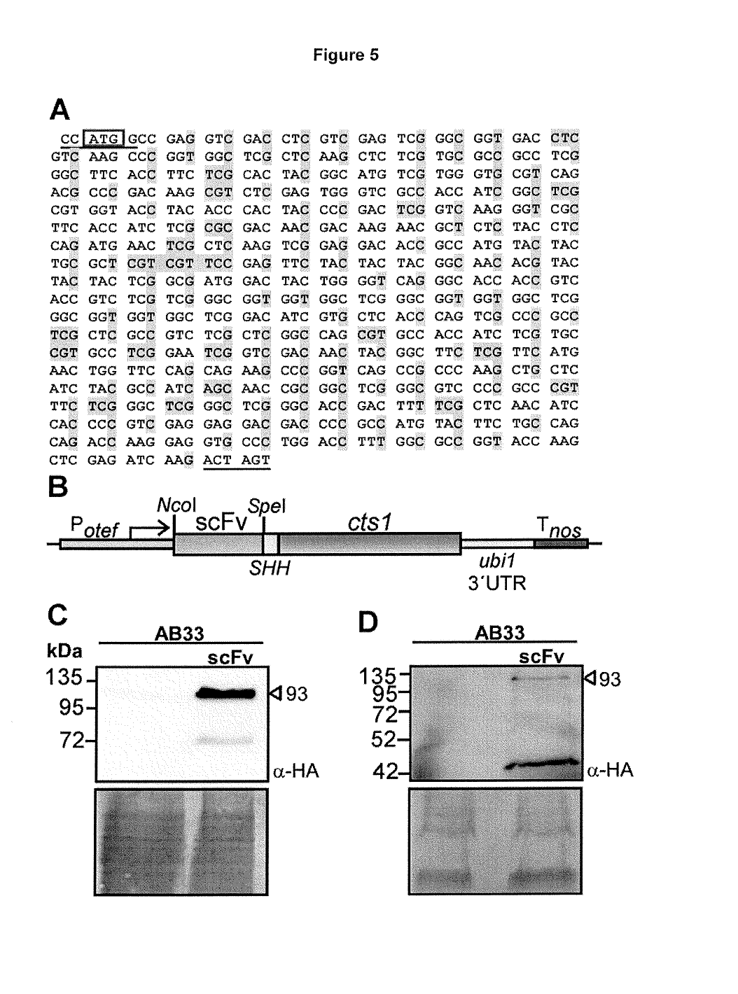

FIG. 5: Single-chain antibodies can be expressed in U. maydis

A, DNA sequence of a synthetic scFv anti-cMyc which was adapted to the context-dependent codon usage of U. maydis. (see SEQ ID No:18 and 19) Bases that were changed are shaded and mostly locate to the wobble position. Restriction sites (NcoI, SpeI) that were introduced for cloning purposes are underlined. The translational start codon ATG is boxed.

B, Schematic representation of the construct encoding the scFv anti-cMyc-SHH-Cts1 fusion protein. See FIG. 4A for further descriptions.

C, Western blot depicting expression of the scFv anti-cMyc-SHH-Cts1 fusion protein migrating slightly above the expected size of about 93 kDa. 10 μg of whole cell protein extracts were analysed with anti-HA antibodies. The Commassie Brilliant Blue stained membrane visualizes equal loading. The expected band size is 93 kDa.

D, Western blot depicting detection of the scFv anti-cMyc-SHH-Cts1 fusion protein in cell-free culture supernatants of filamentous cultures that were enriched by TCA precipitation. AB33 (wt) was used as negative control.

FIG. 6: Cts1 deletion variants

Cts1 deletion variants. Numbers correspond to amino acid positions of Cts1 shown in SEQ ID No:2.

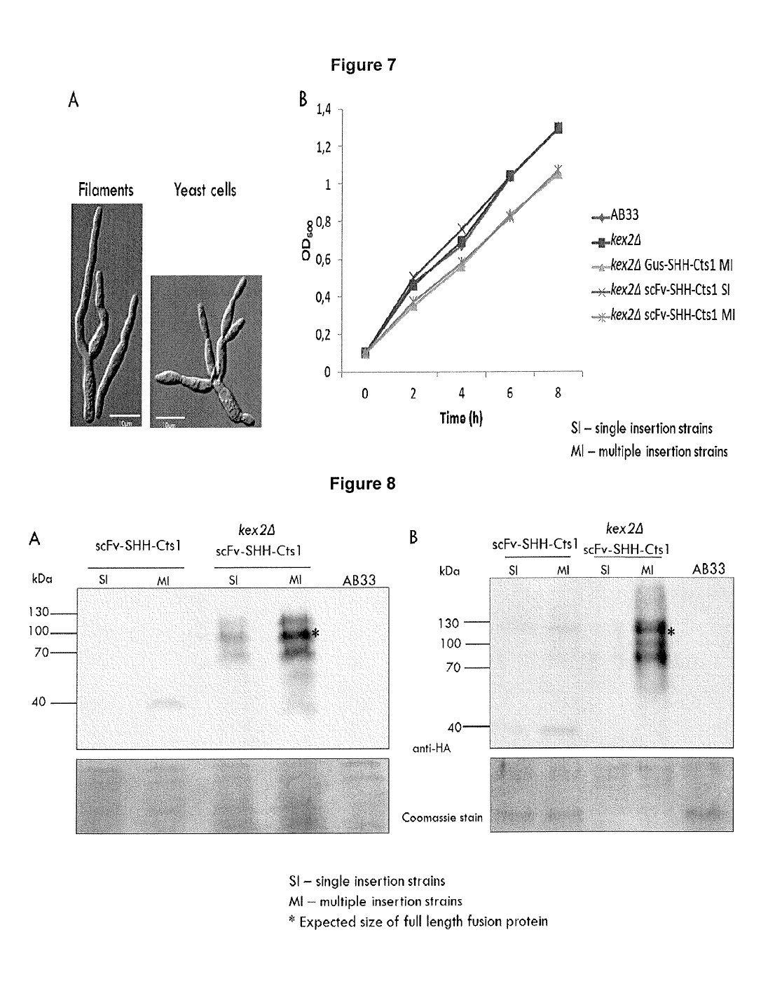

FIG. 7: AB33 kex2Δ strains show an aberrant morphology (yeast cells and filaments) but only a slight decrease in growth rate

A, Morphology of yeast and filamentous kex2 deletion strains.

B, Growth behavior of kex2 deletion strains. The insertion of expression constructs in single copy did not change the growth behavior of the kex2Δ strains. By contrast, multiple insertions of the construct led to a further slight decrease.

FIG. 8: Full length scFv-SHH-Cts1 is present in supernatants of yeast (A) and filamentous (B) AB33 kex2Δ cultures

Asterisks indicate the bands of the predicted size of the scFv-SHH-Cts1 fusion protein.

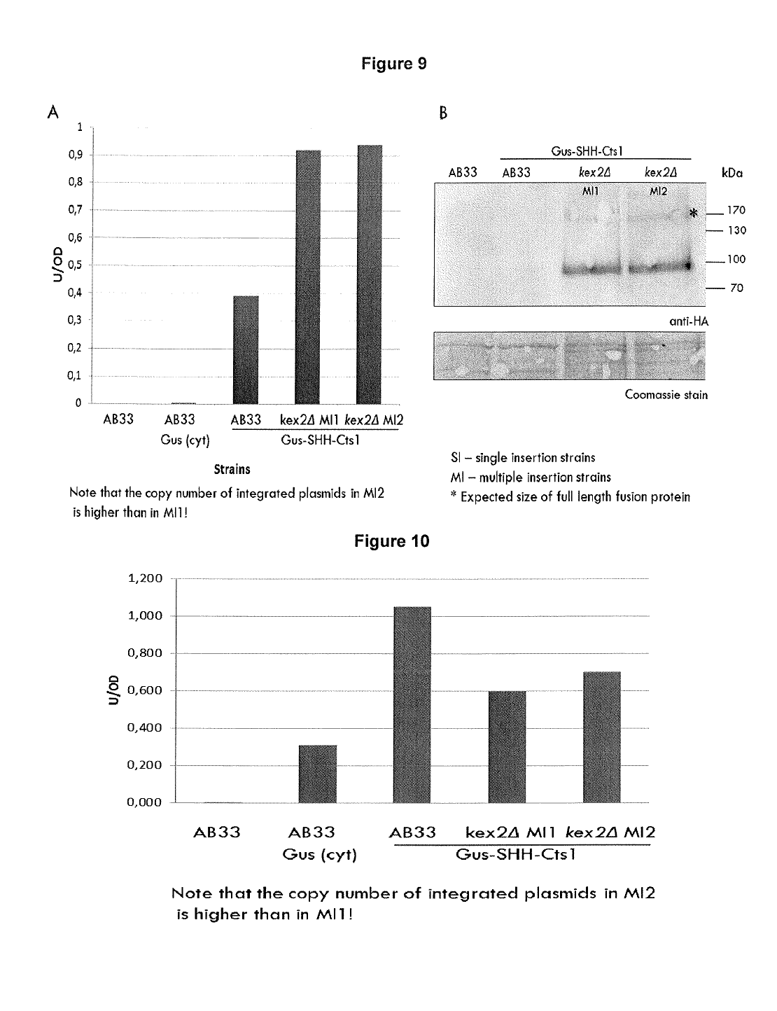

FIG. 9: The yield of Gus-SHH-Cts1 rises in yeast cells of kex2 deletion strains

A, The activity of Gus-SHH-Cts1 in supernatants of yeast cultures rises strongly upon deletion of kex2.

B, A full length band can be detected by Western blot analysis for the first time.

FIG. 10: The activity of Gus-SHH-Cts1 in supernatants of filamentous cultures decreases upon deletion of kex2

FIG. 11: Growth rates (OD_{600 nm}) of different protease deletion strains

For comparison, kex2 deletion strains were added to the graph.

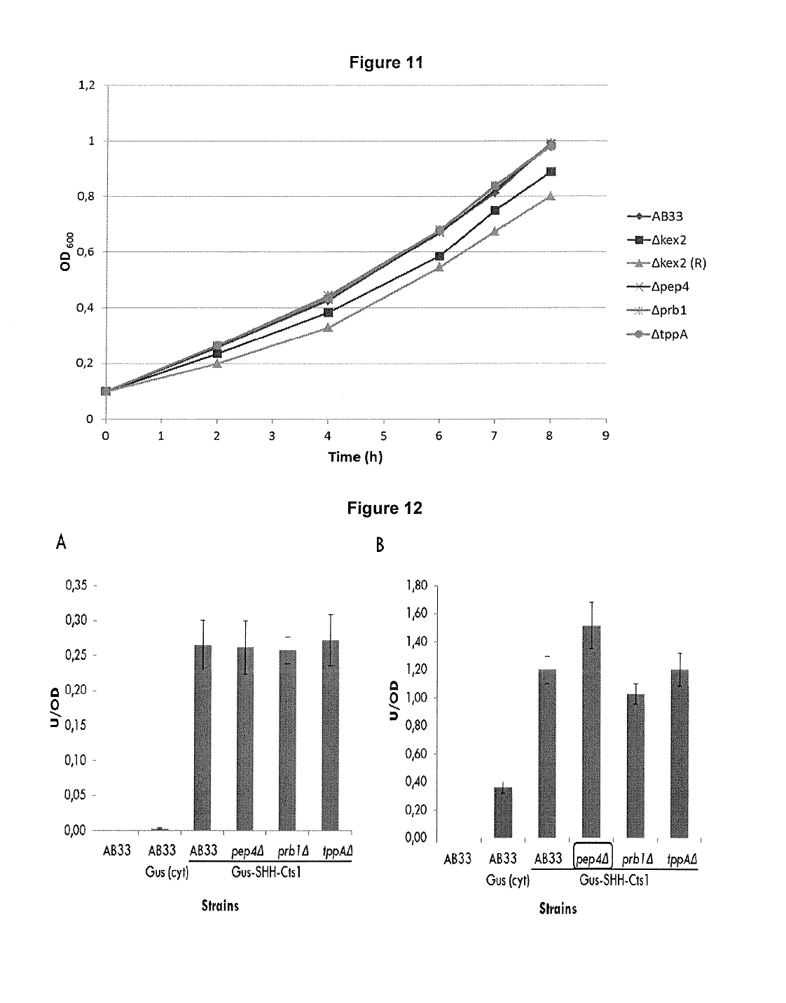

FIG. 12: Gus activity in supernatants of yeast (A) and filamentous (B) cells harboring Gus-SHH-Cts1

Different AB33 derivatives with deletions in individual proteases were tested.

The following Examples illustrate the invention, but are not to be construed as limiting the scope of the invention.

### Materials and Methods

### Strains and Growth Conditions

E. coli K-12 derivate Top10 (Invitrogen/Life Technologies) was used for cloning purposes. Growth conditions for U. maydis strains and source of antibiotics were described previously (Brachmann et al. (2004), cited above). U. maydis strains were generated by transformation of the progenitor strain AB33 with linearised integrative plasmids (see Plasmids and plasmid construction). Homologous integrations at the ip locus were verified by Southern blot analysis using a 2.1 kb probe obtained with the primer combination MF502/MF503 and the template pUMa260 (Brachmann et al. (2004); Loubradou et al. (2001), Mol. Microbiol. 40(3):719-730). Filamentous growth of AB33 derivatives was induced by shifting cells of an exponential growing culture (OD₆₀₀=0.5) from liquid complete medium (C M, Holliday (1974), In King, R. C. (ed.) Handbook of Genetics 1, Plenum Press, New York/USA:5765-595) to nitrate minimal medium (NM; Brachmann et al. (2004), cited above). Cells were incubated at 28° C. shaking with 200 rpm.

### Plasmids and Plasmid Constructions

Standard molecular cloning techniques were followed (Sambrook et al. (2001), cited herein). For PCR, genomic DNA of wild-type strain UM521 (a1b1) was used as a template. Context-dependent codon optimization of gus and the gene for the anti-cMyc scFv was performed as described earlier (Zarnack et al. (2006), Fungal Genet. Biol. 43(11):727-738). The optimized genes were synthesized by Geneart (Invitrogen).

All plasmids generated in this study contain a region encoding an ip allele that confers resistance to the antibiotic carboxin (ip^{r}; Keon et al. (1991), cited above; Broomfield and Hargreaves (1992), cited above). For integration into the ip^{s} locus by homologous recombination, respective plasmids were linearized within the ip^{r} gene using either AgeI or SspI. Subsequently, protoplasts were transformed with the linearized plasmids using selective plates containing carboxin following published methods (Brachmann et al. (2004), cited above).

All integrative vectors for homologous recombination at the ip locus were derived from p123 (Aichinger et al. (2003), Mol Genet. Genomics 270(4):303-314). pCts1-Gus (pUMa1354) was derived from plasmid p123 by replacing egfp with codon-optimized gusA using the NcoI and NotI sites. At the same time the NotI site was replaced by an AscI site which was used together with XbaI to insert the cts1 ORF. pCts1-Gus (pUMa1355) was also derived from plasmid p123 by replacing egfp with codon-optimized gusA and replacement of NotI with AscI like described before. The cts1 ORF was amplified with suitable primers and inserted via XbaI and NcoI. To generate pGus-Cts1-GTH (pUMa1385) the cts1-egfp sequence was extracted from pCts1-Gfp-nat-topo (pUMa828; Koepke et al. (2011), cited herein) by SfiI and AatII restriction and inserted together with a fragment encoding eGfp-TriTap-His by SfiI and AscI obtained from peGfp-TriTag-nos-nat-pBS (pUMa741) to the vector pGus-Cts1 (pUMa1355) linearized with AatII and AscI. To obtain pGus-GTH (pUMa1403), the cts1 ORF in pGus-Cts1-GTH (pUMa1385) was replaced via XbaI and SfiI restriction by a linker obtained by assembly of the primers oSL880 and oSL881. Concomitantly, the XbaI restriction site was replaced with NotI. For generation of pCts1-Gus-GTTH (pUMa1404) a 3.5 kb DNA fragment of pCts1-Gus (pUMa1354) was combined with a 6.8 kb DNA fragment of pGus-GTTH (pUMa1403) via MfeI restriction sites. To obtain pSp-Gus-GTH (pUMa1412) a 3.2 kb fragment was derived from pCts1-Gus-GTH (pUMa1404) by AscI/XbaI restriction and combined with a 5.6 kb fragment derived from pGus-Cts1-GTH (pUMa1385) by AscI/NcoI restriction using a linker generated with the primers oSL968 and oSL969.

For generation of pGus-Cts1-GTH ubi3UTR (pUMa1425), the ubi1 3′UTR and the nos terminator sequence were isolated from pcrg-eGfp-ubi3′UTR-nosT-cbx (pUMa958) and inserted to vector pGus-Cts1-GTH (pUMa1385) via EcoRI/AscI restriction. pGus-Cts1₁₀₃₋₅₀₂-GTH ubi3UTR (pUMa1388) was obtained by amplification of a truncated cts1 version with the primers SL85 and RL293 at the template pGus-Cts1-GTH (pUMa1385). The 1.2 kb PCR product was inserted to pGus-Cts1-GTH ubi3UTR (pUMa1425) using the XbaI and SfiI restriction sites.

For generating pRabX01Gus-SHH-Cts1 ubi3UTR (pUMa1521) the sequence encoding the GTH tag in pGus-Cts1-GTH ubi3UTR was removed by SfiI and AscI restriction and replaced by a fragment coding for the SHH tag generated with suitable primers. A second fragment generated by way of suitable primers was cloned into the vector using the XbaI site. Subsequently, an additional Strep-3HA-10H is tag encoding fragment obtained from pMA_Strep-HA-His (Geneart; pUMa1533) was inserted via SpeI and BspEI. For generation of pRabX1scFv-SHH-Cts1 (pUMa1570), a 767 by NcoI-SpeI fragment of the codon-optimized anti-c-Myc scFv gene obtained from pMK-RQ Um-anti-cMyc-scFv (Geneart; pUMa1465) was inserted between the respective NcoI and SpeI sites of pRabX1Gus-SHH-Cts1 (pUMa1521). All constructions were confirmed by sequencing.

Protein Precipitation from Supernatants of Filamentous Cultures

For the enrichment of Cts1-fusion proteins from culture supernatants, filamentation was induced for six hours. The original protocol (Brachmann et al., 2001) for filament induction was modified such that yeast cells were grown to an OD₆₀₀ of 0.5 and subsequently shifted to an OD₆₀₀ of 1 in nitrate-containing NM medium supplemented with 1.5% (w/v) glucose (Holliday, 1974). Cell-free supernatants were isolated by filtration of the cultures (MN 615% filter paper, Macherey-Nagel) and contained proteins were precipitated using TCA precipitation.

### Western Blot Analysis

Harvested cells were resuspended in 2 ml lysis buffer (100 mM sodium phosphate buffer, pH 8.0; 10 mM Tris/HCl, pH 8.0; 8 M urea; 2× complete protease inhibitor cocktail, Roche), frozen in liquid nitrogen and destroyed in a pebble mill (Retsch; 5 minutes, 30 hz). After centrifugation (6,000 g for 30 minutes at 4° C.) protein concentration of supernatants was determined by Bradford assays (BioRad; Bradford, 1976) and 10 μg total protein was loaded on SDS-PAGE and transferred to a PVDF membrane. Gus reporter proteins were detected using α-Gus (Invitrogen) and α-rabbit IgG HRP conjugates (Cell Signaling) as primary and secondary antibodies, respectively. Cts1 fusion proteins harbouring the SHH tag were detected with primary α-HA antibodies (Roche) and a secondary α-mouse IgG HRP conjugate (H+L; Promega). HRP activity was detected using the ECL plus Western blotting detection system (Amersham Bioscience) and a LAS4000 Mini chemiluminescence imager (Fuji).

### Gus Activity Plate Assay

Gus activity of sporidial cultures was tested by indicator plate assays using CM plates containing 1% (w/v) glucose and the chromogenic substrate X-Gluc (5-bromo-4-chloro-3-indolyl-beta-D-glucuronic acid; 0.5 mg/ml in DMSO). For solvent controls, DMSO was added to the respective plates. Tested strains were grown in liquid CM-glucose medium to an OD₆₀₀ of 0.8. After adjusting the cultures to an OD₆₀₀ of 1, equal volumes were plated and incubated for three days at 28° C.

### Fluorimetric Determination of Gus Activity

Gus activity in culture lysates or supernatants was determined using the specific substrate 4-methylumbelliferyl β-D-galactopyranoside (MUG, Sigma-Aldrich). Culture supernatants of yeast cells as well as filaments induced for six hours (OD600=0.5) were used. Cell-free supernatants were mixed 1:1 with double concentrated Gus assay buffer (10 mM sodium phosphate buffer pH 7.0, 28 μM β-mercaptoethanol, 0.8 mM EDTA, 0.0042% lauroyl-sarcosin, 0.004% Triton-X-100, 2 mM MUG, 0.2 mg/ml (w/v) BSA; prewarmed to 37° C.) and 200 μl aliquots were stopped at 0, 2, 3 and 4.5 h post reaction start with 0.2 mM Na₂CO₃ and stored in the dark (4° C.) until fluorescence was determined in 96-well plates. Relative fluorescence units (RFUs) were determined at 25° C. with excitation and emission wavelengths of 365 nm and 465 nm (gain 60) using a monochromator fluorescence reader (Tecan Safire, Magellan Software). For fluorometric quantitation of MUG conversion to 4-methylumbelliferone (MU), the fluorescent product that is formed in the presence of Gus, a calibration curve was determined using 0, 0.1, 1, 10 and 100 μM MU (Sigma-Aldrich). All activities were determined in technical duplicates.

### Results

### Generation of Reporter Strains to Detect Unconventional Secretion

U. maydis secretes the endochitinase Cts1 (Um10419) that likely functions at the fungal cell wall (Koepke et al. (2011), cited herein). However, according to bioinformatic predictions (e.g., SignalP, http://www.cbs.dtu.dk/services/SignalP/; TMHMM, http://www.cbs.dtu.dk/services/TMHMM/) Cts1 is lacking a conventional N-terminal secretion signal and trans-membrane domains. Thus, secretion is likely to occur through an unconventional mechanism (Nickel and Seedorf (2008), Annu. Rev. Cell. Dev. Bio. 24:287-308) Nickel (2010), Cur. Opin. Biotechnol. 21(5):621-626).

To test this assumption, we developed a reporter system that is based on the cytosolic bacterial enzyme β-Glucuronidase (Gus; Jefferson et al. (1986), Proc. Natl. Acad. Sci. USA 83(22):8447-8451) N-glycosylation of an asparagine residue at position 354 (D₃₅₄) leads to inactivation of Gus (Iturriaga et al. (1989), cited herein). This feature was exploited to discriminate conventional and unconventional secretion: During conventional secretion, Gus passes the endoplasmic reticulum (ER) and the Golgi (Walter and Lingappa (1986), Annuu. Rev. Cell. Biol. 2:499-516) where its eventually modified by N-glycosylation and thus inactivated (Iturriaga et al. (1989), cited herein. In contrast, the enzyme should keep its activity if unconventional secretory routes are taken that avoid ER passage.

Four reporter strains were generated that carry different integrative plasmids (FIG. 1A). Two plasmids code for N- and C-terminal Cts1 fusions to Gus. If Cts1 is secreted unconventionally, active Gus should be co-exported in the respective strains. As controls for cell lysis and conventional secretion, plasmids were generated encoding non-secreted Gus and Sp-Gus (Gus fused to the signal peptide of Um01945, a predicted secreted invertase Suc2), respectively. All constructs carry an additional sequence coding for a C-terminal triple tag consisting of Gfp, Tap and His tag (GTH; FIG. 1A). This should enable the detection and purification of the proteins. All fusion genes were inserted downstream of the constitutively active promoter P_{otef} (Spellig et al. (1996), Mol. Gen. Genet 252:503-509).

The integrative plasmids were used to transform AB33, a strain that allows efficient induction of filamentous growth in nitrate minimal medium (Brachmann et al. (2001), Mol. Microbiol. 42(4):1047-1063). The plasmids were linearized within the ip^{r} allele (e.g., using AgeI; FIG. 1A) and integrated at the ip^{s} locus by homologous recombination (FIG. 1B). The ip^{s} locus codes for the iron-sulfur subunit of the succinyl dehydrogenase (Um00844/Sdh2; Ip^{s}). An amino acid exchange (H₂₅₃L) encoded by the ip^{r} allele mediates carboxin resistance of the enzyme (Ip^{r}; Keon et al. (1991); Broomfield and Hargreaves (1992), both cited above). Thus, carboxin selection of the transformants leads to single- or multi-copy plasmid integration at the ip locus (FIG. 1B). In addition, unwanted ectopic integrations as well as gene conversions occur occasionally. Therefore, all strains were verified by Southern blot analysis (FIG. 1C). In the example AB33 Gus-GTH, two of twelve transformants harbor a single integration and four others carry multiple insertions of the respective integrative plasmid (FIG. 1C), demonstrating the efficiency of this method. The generation of strains containing multiple plasmid insertions leads to increased expression levels, which can be advantageous in biotechnological applications.

### Cts1 is Secreted by an Unconventional Mechanism

The generated reporter strains were used to identify the mode of Cts1 secretion. Firstly, protein expression was verified in Western blot analyses of whole cell extracts (FIG. 2A). Gus fusion proteins were present in the respective strains consistent with their molecular weights (FIG. 2A), whereas AB33 extracts showed only minor background bands, proving the specificity of the antibody. To detect Gus activity we first used indicator plates containing a chromogenic substrate (FIG. 2B). As expected, no staining was detectable for the parental strain AB33 and its derivatives expressing Gus-GTH and Sp-Gus-GTH (FIG. 2B). A faint blue staining was observed for strains expressing Cts1-Gus-GTH. However, expression of Gus-Cts1-GTH led to a strong blue staining surrounding the colonies which suggests that active aglycosylated Gus is secreted to the medium (FIG. 2B). The colonies did not appear blue indicating that the fusion proteins did not attach to the cell wall.

To confirm these results, we next conducted fluorometric Gus assays that allow quantitation of enzymatic activity. As expected, cell extracts of all tested strains with the exception of AB33 displayed Gus activity, confirming that intracellular Gus is active in all strains (FIG. 2C). In cell-free culture supernatants, strains producing Cts1-Gus-GTH or the two control strains showed only background activity (FIG. 2D,E). In contrast, supernatants of Gus-Cts1-GTH strains displayed Gus activity and in this case Gus activity was detected in the supernatants of both yeast (FIG. 2D) and filamentous cultures (FIG. 2E). Notably, due to the different growth modes of yeast and filaments a direct comparison of the Gus activity levels is not applicable. Cell lysis can be excluded as the strain producing Gus-GTH does not display significant Gus activity in culture supernatants. These results are consistent with the indicator plate assay, confirming that N-terminal protein fusions to Cts1 are exported by unconventional secretion. Importantly, this mechanism can be applied to export foreign enzymes in their active form.

### The N-Terminal Cts1 Domain is Dispensable for Secretion

Most commonly protein targeting sequences are present in the N-terminus of proteins (Stroud and Walter (2000), Curr. Opin. Struct. Biol. 9(6):754-759). To address the question whether this holds true for Cts1, an N-terminally truncated protein variant was generated. The rationale for the design of the truncation was based on a sequence comparison of U. maydis Cts1 (UmCts1) to an ortholog, termed SrCts1 (Sr15153; http://mips.helmholtz-muenchen.de/genre/proj/sporisorium; FIG. 3A). The corresponding gene has been identified in the recently sequenced genome of the related fungus Sporisorium reilianum (Schirawski et al. (2010), Science 330(6010):1546-1548. The two proteins share an amino acid identity of 81%. Interestingly, the putative enzymatically active Glyco_{—}18 domain (boxed) displays higher sequence conservation then the remaining parts of the protein. Moreover, there are shorter stretches of high sequence conservation even outside of the Glyco_{—}18 domain at the immediate N-(amino acids 1-34) and C-terminus (amino acids 449-497). (FIG. 3A).

To investigate if the N-terminal part of the protein is essential for secretion of Cts1, a strain expressing Gus-Cts1₁₀₃₋₅₀₂-GTH was generated. Deletion of the amino acids 1-102 of Cts1 in the fusion protein neither affected protein stability (FIG. 3B) nor disturbed protein secretion in yeast cells, as Gus activity could be determined in yeast supernatants at similar levels as for Gus-Cts1-GTH (FIG. 3C). In supernatants of filamentous cultures we also observed Gus activity. This demonstrates that the N-terminal domain is dispensable for Cts1 secretion, suggesting the presence of an unconventional secretion signal.

Further deletion variants can be generated in the same was as described above for the variant lacking amino acids 1-102 of Cts1; see FIG. 6. In particular, either appropriate restriction enzyme recognition sites can be used or the respective deletions are generated by PCR. Resulting constructs are cloned and inserted in the Ustilago maydis genome as described herein.

### Design of an Expression Vector

Attempts to detect or purify full length Cts1-fusion proteins containing the previously described GTH tag from culture supernatants were unsuccessful, probably due to proteolytic cleavage. Thus, a novel expression plasmid was generated that harbours an SHH linker between the gene of interest and cts1 (FIG. 4A). The SHH linker consists of a One-STReP tag (IBA, Göttingen), triple HA tag and a 10×His tag. These small protein extensions should provide flexibility with respect to purification and detection of Cts1 fusion proteins. To test, if protein secretion is increased by enhanced mRNA transport, we inserted the 3′UTR of ubi1, a target transcript of Rrm4 (König et al. (2009), EMBO J. 28, 1855-1866; Koepke et al. (2011), cited herein. Earlier results demonstrated that this sequence contains a functional RNA element that promotes frequency and processivity of microtubule-dependent mRNA transport (König et al., 2009). For testing the improved system Gus was again used as a reporter (FIG. 4A). In a corresponding strain, expression of a Gus-SHH-Cts1 could be confirmed by Western blot analysis of whole cell extracts (FIG. 4B) and furthermore, Gus activity was preserved in yeast culture supernatants (FIG. 4C), demonstrating secretion. In contrast, supernatants of filamentous cells showed about 50% reduction in Gus activity (FIG. 4D). In both experiments, no influence of the ubi1 3′UTR could be detected (FIG. 4C,D). The novel expression vector was designed such that the Gus encoding gene and the SHH linker can be replaced by other genes of choice or linkers containing i.e. protease cleavage sites, respectively, by simple one-step cloning. Thus, this vector is feasible for an application in the expression of biotechnological highly valued proteins (see below).

Expression and Characterization of an Anti-cMyc scFv

To demonstrate that Cts1-mediated secretion can be applied for the export of pharmacological relevant proteins, we aimed to express a single chain antibody (scFv; Bird et al. (1988), Science 242 (4877):423-426) directed against the cMyc epitope EQKLISEEDL of the human oncogene product c-myc as a proof-of-principle. Therefore, a modified version of the gene encoding the anti-cMyc scFv described by Fujiwara et al. (2002), Biochemistry 41:12729-12738 was codon-optimized for U. maydis to avoid premature polyadenylation (Zarnack et al. (2006), cited above; FIG. 5A), inserted into the expression vector pRabX1 (FIG. 5B) and AB33 derivatives harbouring this plasmid were generated as described above. Western blot analysis using whole cell extracts confirmed that the scFv-SHH-Cts1 fusion protein is produced (FIG. 5C), migrating at the expected size of 93 kDa. The new architecture of the fusion protein enabled detection of the full length fusion protein in cell-free culture supernatants of filamentously growing cells (FIG. 5D). In essence, the successful expression of the single-chain antibody as fusion proteins constitutes the first important step towards production of pharmacological relevant proteins.

### Deletion of the Central Protease Kex2

In other organisms, e.g. Saccharomyces cerevisiae, the serine protease Kex2 has been identified as an activator of various secreted and cell wall-associated enzymes or proteins. It is also known that secreted proteases are targeted by Kex2, which resides in the trans-Golgi network and removes the pro-sequence from the N-terminus of protease precursors in transit by mostly acting on (di)basic protease cleavage sites (e.g. KR or RR). This modification leads to the activation of the respective proteases. Thus, by deletion of kex2, different secreted proteases cannot be activated anymore and the proteolytic activity in culture supernatants is likely getting reduced.

The kex2 deletion was performed in the AB33 background using homologous recombination as is known in the art for Ustilago maydis. Therefore, the corresponding gene was completely removed and replaced by a hygromycin resistance cassette. Correct mutants were confirmed by Southern blot analysis.

Yeast and filamentous AB33 kex2Δ strains display a strong phenotype that discriminates them from the parental strain AB33: yeast cells form aggregates in liquid culture and the microscopic observation of the cell morphology shows aberrant cell shapes and a cytokinesis defect. However, growth rates of yeast cells are comparable to the parental strain AB33 (FIG. 7B). kex2Δ filaments are growing mostly unipolar, but are odd shaped (thicker than wild type cells) and relatively short (FIG. 7A).

To analyze the yield of unconventionally secreted proteins in culture supernatants of kex2Δ strain, expression cassettes coding for either an anti-myc scFv-SHH-Cts1 or a Gus-SHH-Cts1 fusion protein were introduced into this strain background. Single insertion mutants did show only a minor growth rate reduction (comparable to the AB33 kex2Δ strain lacking an expression cassette), but upon insertion of multiple copies of the expression construct a slightly higher growth rate reduction was observed (FIG. 7B).

To analyse the effect of the kex2 deletion with respect to the yield of secreted proteins exported by unconventional secretion, supernatants of the scFv-SHH-Cts1-expressing strain were subjected to Western blot analysis. In strong contrast to the corresponding AB33 derivative that still expresses kex2 (AB33 scFv-SHH-Cts1), the deletion strain (AB33kex2Δ scFv-SHH-Cts1) allowed detection of full length scFv-SHH-Cts1 (about 92.6 kDa) in culture supernatants of both yeast cell and filamentous cultures (FIG. 8A,B). Multiple insertion of the expression construct led to detection of stronger signals, indicating that the use of multiple insertions might be useful to increase protein yield. These results are a clear indication, that proteolytic degradation is strongly reduced in the kex2Δ background.

To further analyse the effect of the kex2 deletion on the yields of active protein, Gus-Cts1 fusions were used as a quick read-out. To this end, the strain AB33kex2Δ Gus-SHH-Cts1 was generated and fluorometric Gus assays were performed using yeast and filamentous culture supernatants (FIG. 9A). Yeast cell supernatants showed a strong increase in Gus activity (by about 135%) in the absence of the kex2 protease compared to the progenitor strain (OD₆₀₀ of 0.7; FIG. 9A). Furthermore, a faint signal for the full length fusion protein (Gus-SHH-Cts1; FIG. 9B) could now be observed in Western blot experiments for the first time, along with a thicker band of lower size (probable degradation product). AB33 (harboring no Gus) and AB33 expressing intracellular Gus (indicated as Gus(cyt)) were used as controls.

For filamentous cultures, in contrast, a strong reduction of the Gus activity was determined in the absence of Kex2 (FIG. 10), suggesting that the kex2 deletion has a negative effect on protein yield, likely due to the strong morphologic changes during filament induction which could influence the unconventional secretion apparatus. Again, AB33 (harboring no Gus) and AB33 expressing intracellular Gus were used as controls.

In sum, the deletion of the central activator protease kex2 led to a significant increase in protein yield using secretion via the unconventional pathway in which Cts1 deals as a carrier.

### Deletion of Further Proteases

Based on bioinformatic analyses, at least 31 proteases are encoded in the U. maydis genome (MUMDB; http://mips.helmholtz-muenchen.de/genre/proj/ustilago/). According to literature, proteolytic degradation in culture supernatants of filamentous fungi (e.g. Aspergillus oryzae) is often due to a limited number of proteases (here termed “key proteases”). 3 proteases were picked because respective strains showed the best effects with respect to protein yield after their deletion in other fungi. The homologs were identified in the U. maydis genome (a predicted secreted aspartic protease Um04926, designated Pep4; a predicted lysosomal serine protease Um04400, designated Prb1 and a predicted lysosomal tripeptidyl peptidase Um06118, designated TppA) and deleted in the AB33 Gus-SHH-Cts1 background, to gain a read-out for the protein yield in supernatants. Gus activity was then measured in the respective strains using supernatants of the yeast and filamentous forms. Importantly, growth rates (yeast cell cultures) are not affected for the 3 protease deletion strains (FIG. 11). The filamentous growth of prb1Δ strains was strongly reduced (not shown), whereas hyphae formation of the other deletion strains seemed normal.

The individual deletion of the 3 proteases had no effect on Gus activity in yeast cell supernatants (FIG. 12A). However, in supernatants of filamentous cultures, the pep4Δ strain showed a significant increase (about 30%) of Gus activity compared to the progenitor strain (FIG. 12B). In sum, pep4Δ strains displayed a slight increase of protein yield during filamentous growth.

1. An expression cassette comprising
(a) a nucleotide sequence encoding
(i) an amino acid sequence having amino acids n-502 of the amino acid sequence shown in SEQ ID No:2, wherein n is amino acid position 1 of SEQ ID No:2, or a fragment thereof which directs unconventional protein secretion, or
(ii) an amino acid sequence which is at least 60% identical to the amino acid sequence of (i) and which directs unconventional protein secretion; and
(b) a nucleotide sequence encoding a protein of interest,
wherein nucleotide sequence (a) and (b) are fused in frame,
with the proviso that nucleotide sequence (b) does not encode green fluorescence protein.

2. The expression cassette of claim 1, wherein n is an integer in the range of amino acid position 43 to amino acid position 461 of SEQ ID No:2.

3. The expression cassette of claim 1, wherein n is an integer in the range of amino acid position 103 to amino acid position 461 of SEQ ID No:2.

4. The expression cassette of claim 1, wherein n is an integer in the range of amino acid position 235 to amino acid position 461 of SEQ ID No:2.

5. The expression cassette of claim 1, wherein n is an integer in the range of amino acid position 319 to amino acid position 461 of SEQ ID No:2.

6. The expression cassette of claim 1, wherein n is amino acid position 461 of SEQ ID No:2.

7. The expression cassette of claim 1, wherein nucleotide sequence (a) lacks the nucleotide sequence encoding amino acids 104-460, 200-232, 237-247 and/or 319-328 of the amino acid sequence shown in SEQ ID No:2.

8. The expression cassette of claim 1, wherein nucleotide sequence (a) comprises
(i) amino acids 43-502 of the amino acid sequence shown in SEQ ID No:2
(ii) amino acids 103-502 of the amino acid sequence shown in SEQ ID No:2,
(iii) amino acids 235-502 of the amino acid sequence shown in SEQ ID No:2,
(iv) amino acids 319-502 of the amino acid sequence shown in SEQ ID No:2, or
(v) amino acids 461-502 of the amino acid sequence shown in SEQ ID No:2.

9. The expression cassette of claim 1, further comprising one or more nucleotide sequence(s) (c) fused to the 5′- and/or 3′-end of the nucleotide sequence (a) and/or (b).

10-11. (canceled)

12. The expression cassette of claim 1, wherein the nucleotide sequence (a), (b) and/or (c) comprise(s) one or more further nucleotide sequence(s) (d) fused to the 5′- and/or 3′-end of the nucleotide sequence (a), (b) and/or (c).

13-20. (canceled)

21. The expression cassette of claim 1, wherein said nucleotide sequence (c) can be bound by a polypeptide comprising at least one sequence specific RNA binding domain.

22-26. (canceled)

27. A vector comprising the expression cassette of claim 1.

28. A host cell comprising the expression cassette of claim 1 or the vector of claim 27.

29-31. (canceled)

32. A method for the production of the host cell of claim 28, comprising transforming a host cell with the expression cassette of claim 1 or the vector of claim 27.

33. A method for the production of a polypeptide comprising
(a) culturing the host cell of claim 28 to allow expression of said polypeptide;
(b) harvesting said polypeptide from the culture medium or host cell.

34. Use of the expression cassette of claim 1 or the vector of claim 27 for the production of a recombinant host cell.

35. Use of the expression cassette of claim 1, the vector of claim 27 or the host cell of claim 28 for the production of a polypeptide.

36. A kit (expression system) comprising the expression cassette of claim 1, the vector of claim 27 and/or the host cell of claim 28, and optionally means for transforming a host cell, a host cell, culture medium and/or an antibiotic for selecting and/or growing transformed host cells.

37. A method for identifying an amino acid sequence which directs unconventional protein secretion, comprising
(a) providing a host cell expressing a fusion protein comprising (i) an amino acid sequence which is suspected to direct unconventional protein secretion and (ii) an amino acid sequence encoding a marker protein having a detectable activity which is subject to N-glycosylation in said host cell, thereby inactivating said marker protein,
(b) determining whether said marker protein is secreted by said host cell by detecting its activity,
wherein said amino acid sequence which is suspected to direct unconventional protein secretion directs unconventional protein secretion if it is active after secretion by said host cell.

38. (canceled)

39. Use of β-glucuronidase (Gus) for the identification of an amino acid sequence which directs unconventional protein secretion.
